(19) United States
(12) Patent Application Publication
(10) Pub. No.: US 2006/0029665 A1
(43) Pub. Date: Feb. 9, 2006

(54) Pilocarpine compositions and methods of use thereof

(75) Inventor: Nikhilesh N. Singh, Mill Valley, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: TransOral Pharmaceuticals, Inc., Corte Madera, CA (US)

(21) Appl. No.: 11/195,567

(22) Filed: Aug. 1, 2005

Related U.S. Application Data
(60) Provisional application No. 60/598,625, filed on Aug. 3, 2004.

(51) Int. Cl. A61K 9/20; A61K 36/185

(52) U.S. Cl. 424/464; 424/774

(57) ABSTRACT

The present invention provides novel compositions for the delivery of pilocarpine or a pharmaceutically acceptable salt thereof across the oral mucosa, preferably across the buccal mucosa. In particular, the buffer systems in the compositions of the present invention contain an amount of a strong base that is less than the amount of a weak base, thereby increasing the stability of compositions such as chewing gum compositions and raising the pH of saliva to a pH greater than about 7.5 to facilitate the substantially complete conversion of pilocarpine from its ionized to its un-ionized form. Methods for using the compositions of the present invention for treating conditions such as dry mouth are also provided.

### CROSS-REFERENCE TO RELATED APPLICATION

This application claims priority to U.S. Ser. No. 60/598,625 filed Aug. 3, 2004 (Atty Docket No. 022205-000700US), the disclosure of which is incorporated herein by reference.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Dry mouth, known medically as xerostomia, is a condition that affects a person's ability to produce saliva. Such salivary gland hypofunction is typically caused by medication such as decongestants, diuretics, and antihistamines; systemic diseases such as autoimmune diseases (e.g., Sjögren's syndrome, rheumatoid arthritis), anemia, and diabetes; or medical therapy such as radiotherapy for head and neck cancers. Pilocarpine, a naturally-occurring alkaloid obtained from plants of the genus Pilocarpus, has proven useful in treating dry mouth. Pilocarpine is thought to act as a stimulant of the parasympathetic nervous system by binding to acetylcholine receptors and promotes the flow of saliva and urine and increases perspiration. Because pilocarpine increases the outflow of fluid from the eye, reduces the pressure within the eye, and causes the pupil to contract, the drug is also used to treat some types of glaucoma.

Typically, pilocarpine is delivered in the form of an oral dosage such as a tablet or capsule that is swallowed. For example, Salagen® (MGI Pharma, Inc.; Bloomington, Minn.) is a tablet for oral administration containing pilocarpine hydrochloride. However, the delivery of pilocarpine via oral administration has several disadvantages, including drug losses during hepatic first pass metabolism, during chemical and enzymatic degradation within the gastrointestinal tract, and during absorption. These drug losses not only increase the variability in drug response, but also often require that the medicament be given in greater initial doses. In addition, because the drug has to pass through the gastrointestinal system in order to enter the blood stream, the time to reach a therapeutic effect may be quite long, typically around forty-five minutes or longer.

Accordingly, other routes of pilocarpine administration have been investigated, including those involving transport across the mucous membranes. Of the various mucous membranes (e.g., oral, rectal, vaginal, ocular, nasal, etc.), drug delivery via the mucous membranes in the oral cavity seems to be the most easily tolerated by patients. In addition to avoiding the problems with traditional oral administration, drug delivery via the mucous membranes of the oral cavity has certain other advantages, due to the properties of the oral mucosa itself. For example, the mucous membranes of the oral cavity are highly vascularized and well supplied with lymphatic drainage sites.

In general, the mucous membranes of the oral cavity can be divided into five main regions: the floor of the mouth (sublingual), the cheeks (buccal), the gums (gingival), the roof of the mouth (palatal), and the lining of the lips. These regions differ from each other with respect to their anatomy, drug permeability, and physiological response to drugs. For example, in terms of permeability, sublingual is more permeable than buccal, which is more permeable than palatal. This permeability is generally based on the relative thickness and degree of keratinization of these membranes, with the sublingual mucosa being relatively thin and non-keratinized, the buccal mucosa being thicker and non-keratinized, and the palatal mucosa being intermediate in thickness, but keratinized.

In addition to the differences in permeability of the various mucous membranes, the extent of drug delivery is also affected by the properties of the drug to be delivered. The ability of a molecule to pass through any mucous membrane is dependent upon its size, its lipid solubility, and the extent to which it is ionized, among other factors.

The extent to which a drug is ionized has further been investigated with respect to drug delivery across the mucous membranes. Ionization is dependent on the dissociation constant, or pKa of the molecule, and the pH of the molecule's surrounding environment. In its un-ionized form, a drug is sufficiently lipophilic to traverse a membrane via passive diffusion. In fact, according to the pH partition hypothesis, only un-ionized, non-polar drugs will penetrate a lipid membrane.

At equilibrium, the concentrations of the un-ionized form of the drug are equal on both sides of the membrane. As the concentration gradient drives passive diffusion, an increase in the percentage of the un-ionized form of a drug correspondingly increases the transmucosal absorption of the drug. Maximum absorption across the membrane is thought to occur when a drug is 100% in its un-ionized form. Similarly, absorption across the membrane decreases as the extent of ionization increases. Therefore, one may influence the extent of drug absorption across the mucous membranes of the oral cavity by altering the salivary pH.

For example, U.S. patent application Ser. No. 10/113,088 describes a chewing gum composition for enhancing pilocarpine absorption across the buccal cavity by raising salivary pH through the use of a binary basic buffer system, in which the amount of a strong base is greater than the amount of a weak base. However, such chewing gum compositions lack stability and liquefy in the mouth upon administration. As such, binary basic buffer systems containing a greater amount of a strong base than a weak base have reduced utility for delivering pilocarpine across the oral mucosa.

Accordingly, there is a need in the art for compositions with increased stability for delivering pilocarpine across the oral mucosa having buffer systems that facilitate absorption of the drug. Similarly, there is a need in the art for compositions with increased stability for delivering pilocarpine across the oral mucosa having a buffer system that produces a final pH, independent of the initial pH, and sustains that final pH for a given period of time. In addition, there is a need in the art for compositions with increased stability that are capable of rapidly facilitating substantially complete conversion of pilocarpine from its ionized to its un-ionized form. The present invention satisfies these and other needs.

### BRIEF SUMMARY OF THE INVENTION

The present invention provides novel compositions for the delivery of pilocarpine or a pharmaceutically acceptable salt thereof across the oral mucosa. In particular, the buffer systems in the compositions of the present invention contain an amount of a strong base that is less than the amount of a weak base. Such buffer systems advantageously increase the stability of compositions such as chewing gum compositions and raise the pH of saliva to a pH greater than about 7.5, thereby facilitating the substantially complete conversion of pilocarpine from its ionized to its un-ionized form. As a result, the dose of pilocarpine administered is rapidly and efficiently absorbed by the oral mucosa. Furthermore, delivery of pilocarpine across the oral mucosa bypasses hepatic first pass metabolism of the drug and avoids chemical and enzymatic degradation of the drug within the gastrointestinal tract. Methods for using the compositions of the present invention for treating conditions such as dry mouth are also provided.

As such, in one aspect, the present invention provides a solid dosage form composition for delivery of pilocarpine across the oral mucosa, the composition comprising:
(a) pilocarpine or a pharmaceutically acceptable salt thereof;
(b) a carrier; and
(c) a binary buffer system comprising a strong base and a weak base, wherein the amount of the strong base is less than the amount of the weak base,
   wherein the binary buffer system raises the pH of saliva to a pH greater than about 7.5, irrespective of the starting pH of saliva.

In another aspect, the present invention provides a composition for delivery of pilocarpine across the oral mucosa, the composition comprising:
(a) pilocarpine or a pharmaceutically acceptable salt thereof;
(b) a carrier; and
(c) a binary buffer system comprising a strong base and a weak base,
   wherein the amount of the strong base is less than the amount of the weak base.

In yet another aspect, the present invention provides a composition for delivery of pilocarpine across the oral mucosa, the composition comprising:
(a) pilocarpine or a pharmaceutically acceptable salt thereof;
(b) a carrier; and
(c) a binary buffer system comprising a strong base or a weak base and a second buffering agent.

In still yet another aspect, the present invention provides a composition for delivery of pilocarpine across the oral mucosa, the composition comprising:
(a) pilocarpine or a pharmaceutically acceptable salt thereof;
(b) a carrier; and
(c) a binary buffer system comprising a metal oxide and a citrate, phosphate, or borate salt.

In a further aspect, the present invention provides a composition for delivery of pilocarpine across the oral mucosa, the composition comprising:
(a) pilocarpine or a pharmaceutically acceptable salt thereof;
(b) a carrier; and
(c) a ternary buffer system comprising a strong base, a weak base, and a third buffering agent.

In another aspect, the present invention provides a composition for delivery of pilocarpine across the oral mucosa, the composition comprising:
(a) pilocarpine or a pharmaceutically acceptable salt thereof;
(b) a carrier; and
(c) a buffer system comprising a strong base or a weak base and two or more buffering agents selected from the group consisting of a metal oxide, a citrate salt, a phosphate salt, and a borate salt.

In yet another aspect, the present invention provides a method for treating dry mouth in a subject in need thereof, the method comprising:
administering to the subject a composition comprising a therapeutically effective amount of pilocarpine or a pharmaceutically acceptable salt thereof; a carrier; and a binary buffer system comprising a strong base and a weak base, wherein the amount of the strong base is less than the amount of the weak base.

Other objects, features, and advantages of the present invention will be apparent to one of skill in the art from the following detailed description and figures.

### BRIEF DESCRIPTION OF THE DRAWINGS

FIG. 1 shows the mean saliva output over time for an inventive pilocarpine chewing gum composition as compared to a dose equivalent commercial oral tablet (Salagen®). * denotes statistical significance.

### DETAILED DESCRIPTION OF THE INVENTION

### I. DEFINITIONS

As used herein, the following terms have the meanings ascribed to them unless specified otherwise.

The term “xerostomia” refers to a dryness of the mouth resulting from reduced or absent saliva flow. Xerostomia is typically characterized by symptoms including, without limitation, taste disorders (dysgeusia), a painful tongue (glossodynia), an increased need to drink water, increased dental caries, parotid gland enlargement, inflammation and fissuring of the lips (cheilitis), inflammation or ulcers of the tongue and buccal mucosa, oral candidiasis, salivary gland infection (sialadenitis), halitosis, and cracking and fissuring of the oral mucosa.

The term “Sjögren's syndrome” refers to a chronic disease in which white blood cells attack the moisture-producing glands such as salivary glands. Sjögren's syndrome is typically characterized by symptoms including, without limitation, dryness of the mouth, dryness of the eyes, dryness of organs such as the kidneys, gastrointestinal tract, blood vessels, lung, liver, pancreas, and central nervous system, fatigue, and joint pain.

The term “mucositis” refers an inflammation and ulceration of the lining of the mouth, throat, or gastrointestinal tract and is commonly associated with chemotherapy or radiotherapy for cancer. Mucositis is typically characterized by symptoms including, without limitation, dryness of the mouth, redness and swelling of the gums, and ulcerations in the mouth and throat.

The term “stomatitis” refers to an inflammation of the mucous lining of any of the structures in the mouth, which may involve the cheeks, gums, tongue, lips, and roof or floor of the mouth. Stomatitis is typically characterized by symptoms including, without limitation, dryness of the mouth, painful ulcers that are usually located on the lips, cheeks, gums, or roof or floor of the mouth, halitosis, and pain, redness, swelling, and occasional bleeding from the affected area.

The terms “therapeutic agent” and “drug” are used interchangeably herein to refer to a substance having a pharmaceutical, pharmacological, psychosomatic, or therapeutic effect. Preferably, the therapeutic agent or drug is pilocarpine, e.g., in its free base form, or a pharmaceutically acceptable salt thereof. Suitable pharmaceutically acceptable salts of pilocarpine include, without limitation, pilocarpine hydrochloride, pilocarpine nitrate, pilocarpine sulfate, pilocarpine acetate, pilocarpine citrate, pilocarpine tartrate, pilocarpine zinc chloride monohydrate, pilocarpine salicylate, a concentrated extract of Pilocarpus leaves, and combinations thereof. In a particularly preferred embodiment, the therapeutic agent is pilocarpine hydrochloride.

The term “therapeutically effective amount” refers to the amount of pilocarpine or a pharmaceutically acceptable salt thereof that is capable of achieving a therapeutic effect in a subject in need thereof. For example, a therapeutically effective amount of pilocarpine or a pharmaceutically acceptable salt thereof can be the amount that is capable of preventing or relieving one or more symptoms associated with dry mouth.

The term “bioavailability” refers to the rate and/or extent to which a drug is absorbed or becomes available to the treatment site in the body.

As used herein, the phrase “substantially complete conversion of pilocarpine from its ionized to its un-ionized form” refers to greater than about 50% conversion of pilocarpine from its ionized form into its un-ionized form. For example, the buffer system may favor at least about 50%, 55%, 60%, 65%, 70%, 75%, 80%, 85%, 90%, 95%, or 99% conversion of pilocarpine from its ionized form into its un-ionized form. In some embodiments, the conversion occurs within about 10 minutes, e.g., within about 1, 2, 3, 4, 5, 6, 7, 8, 9, or 10 minutes, following administration.

The term “gum base” refers to an elastomeric non-soluble primary base material used to manufacture chewing gum. Suitable gum base materials for use in the present invention include, without limitation, materials selected from among the many water-insoluble and saliva-insoluble gum base materials known in the art. In certain instances, the gum base comprises at least one hydrophobic polymer and at least one hydrophilic polymer. Non-limiting examples of suitable hydrophobic and hydrophilic polymers for gum bases include both natural and synthetic polymers such as elastomers, rubbers, and combinations thereof. In certain other instances, the gum base is a commercially available gum base, e.g., Pharmagum™ M, S, or C (SPI Pharma Group; New Castle, Del.). Pharmagum™ gum bases typically comprise a mixture of gum base (e.g., butyl rubber material), sweetening agent, plasticizer, and sugar.

The term “administering” refers to administration of the compositions of the present invention to the mucous membranes of the oral cavity (i.e., oral mucosa). Examples of suitable sites of administration within the oral mucosa include, without limitation, the mucous membranes of the floor of the mouth (sublingual mucosa), the cheeks (buccal mucosa), the gums (gingival mucosa), the roof of the mouth (palatal mucosa), the lining of the lips, and combinations thereof. Preferably, the compositions of the present invention are administered to the buccal mucosa, sublingual mucosa, or a combination thereof.

### II. GENERAL

The present invention provides novel compositions for the delivery of pilocarpine or a pharmaceutically acceptable salt thereof across the oral mucosa, preferably across the buccal mucosa. In particular, the buffer systems in the compositions of the present invention contain an amount of a strong base that is less than the amount of a weak base, thereby increasing the stability of compositions such as chewing gum compositions and raising the pH of saliva to a pH greater than about 7.5 to facilitate the substantially complete conversion of pilocarpine from its ionized to its un-ionized form. Furthermore, delivery of pilocarpine across the oral mucosa advantageously bypasses hepatic first pass metabolism of the drug and avoids enzymatic degradation of the drug within the gastrointestinal tract. As a result, the bioavailability of pilocarpine is increased, thereby reducing the time to onset of therapeutic activity as compared to traditional dosage forms for oral (e.g., tablet) administration. Methods for using the compositions of the present invention for treating conditions such as dry mouth are also provided.

The present invention is based upon the surprising discovery that pilocarpine chewing gum compositions containing a binary buffer system, in which the amount of a strong base is less than the amount of a weak base, have markedly increased in vitro (i.e., shelf-life) and in vivo (i.e., cud size) stability profiles as compared to a similar pilocarpine chewing gum composition described in U.S. patent application Ser. No. 10/113,088, in which the amount of a strong base is greater than the amount of a weak base. In fact, it was counterintuitive to expect that the addition of a greater amount of a weak base, which is more hygroscopic than a strong base, would provide the markedly increased in vitro and in vivo stability profiles observed with the compositions of the present invention. For example, as shown in Example 1 below, the inventive pilocarpine chewing gum compositions are stable for at least 3 months at either 25° C. or 30° C., while the chewing gum composition described in U.S. patent application Ser. No. 10/113,088 begins to decompose within the first month and is discarded within the next two months. Furthermore, the inventive pilocarpine chewing gum compositions produce a large sized cud upon mastication, while the pilocarpine chewing gum composition described in U.S. patent application Ser. No. 10/113,088 liquefies upon mastication and produces little to no cud. As such, the inventive pilocarpine chewing gum compositions are capable of providing exceptional mouth-feel physical properties, chewing texture, and stability, resulting in increased patient compliance.

### III. DESCRIPTION OF THE EMBODIMENTS

In one aspect, the present invention provides a solid dosage form composition for delivery of pilocarpine across the oral mucosa, the composition comprising:
(a) pilocarpine or a pharmaceutically acceptable salt thereof;
(b) a carrier; and
(c) a binary buffer system comprising a strong base and a weak base, wherein the amount of the strong base is less than the amount of the weak base,
   wherein the binary buffer system raises the pH of saliva to a pH greater than about 7.5, irrespective of the starting pH of saliva.

In one embodiment, the binary buffer system raises the pH of saliva to a pH of from about 8.0 to about 10.0, e.g., about 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, irrespective of the starting pH of saliva. In another embodiment, the pharmaceutically acceptable salt of pilocarpine is selected from the group consisting of pilocarpine hydrochloride, pilocarpine nitrate, pilocarpine sulfate, pilocarpine acetate, pilocarpine citrate, pilocarpine tartrate, pilocarpine zinc chloride monohydrate, pilocarpine salicylate, a concentrated extract of Pilocarpus leaves, and combinations thereof. Preferably, the pharmaceutically acceptable salt of pilocarpine is pilocarpine hydrochloride.

In another embodiment, the strong base is a carbonate salt. Preferably, the carbonate salt is selected from the group consisting of sodium carbonate, potassium carbonate, calcium carbonate, ammonium carbonate, and magnesium carbonate. In yet another embodiment, the weak base is a bicarbonate salt. Preferably, the bicarbonate salt is selected from the group consisting of sodium bicarbonate, potassium bicarbonate, calcium bicarbonate, ammonium bicarbonate, and magnesium bicarbonate. In a particularly preferred embodiment, the strong base is sodium carbonate and the weak base is sodium bicarbonate. In some preferred embodiments, the weight ratio of carbonate salt to bicarbonate salt is at least about 1:3, preferably from about 1:3 to about 1:10, more preferably from about 1:4 to about 1:6, and still more preferably about 1:5. In other preferred embodiments, the amount of the strong base is sufficiently less than the amount of the weak base to retain the solid dosage form for at least 3 months (e.g., 6 months) at room temperature.

In yet another embodiment, the compositions of the present invention are in a dosage form selected from the group consisting of a chewing gum, a lozenge, a chewable tablet, and a dissolving tablet such as a slow-dissolving tablet or a quick-dissolving tablet. Preferably, the composition is a chewing gum. A description of chewing gum compositions containing pilocarpine is provided in Example 2 below.

In a preferred embodiment, pilocarpine is delivered across an oral mucosa selected from the group consisting of the buccal mucosa, the sublingual mucosa, and a combination thereof. In a particularly preferred embodiment, the composition (e.g., chewing gum) is administered buccally so that pilocarpine is delivered across the buccal mucosa.

In another embodiment, the carrier is typically a solid, semi-solid, or liquid such as a gum base, a binder, or combinations thereof. Suitable gum bases for use in the compositions of the present invention include, for example, materials selected from among the many water-insoluble and saliva-insoluble gum base materials known in the art. In certain instances, the gum base comprises at least one hydrophobic polymer and at least one hydrophilic polymer. Non-limiting examples of suitable hydrophobic and hydrophilic polymers for gum bases include both natural and synthetic polymers such as elastomers, rubbers, and combinations thereof. Examples of suitable natural polymers include, without limitation, substances of plant origin such as chicle, jelutong, gutta percha, crown gum, and combinations thereof. Examples of suitable synthetic polymers include elastomers such as butadiene-styrene copolymers, isobutylene and isoprene copolymers (e.g., “butyl rubber”), polyethylene, polyisobutylene, polyvinylester (e.g., polyvinyl acetate and polyvinyl acetate phthalate), and combinations thereof. In other instances, the gum base comprises a mixture of butyl rubber (i.e., isobutylene and isoprene copolymer), polyisobutylene, and optionally, polyvinylacetate (e.g., having a molecular weight of approximately 12,000). In certain instances, the inclusion of a hydrophilic polymer such as polyvinylacetate to a butyl rubber-based gum base can further act synergistically on the absorption of the therapeutic agent. In a preferred embodiment, the gum base is a commercially available gum base, e.g., Pharmagum™ M, S, C, or combinations thereof. The gum base typically comprises from about 40 to about 90 weight percent of the composition, and preferably from about 70 to about 80 weight percent of the composition.

Suitable binders for use in the compositions of the present invention include, without limitation, sugar alcohols such as mannitol, sorbitol, and xylitol; sugars such as lactose, dextrose, sucrose, glucose, and powdered sugar; natural gums such as acacia gum, xanthan gum, guar gum, tara gum, mesquite gum, fenugreek gum, locust bean gum, ghatti gum, and tragacanth gum; other substances such as inositol, molasses, maltodextrin, starch, cellulose, microcrystalline cellulose, polyvinylpyrrolidone, alginate, extract of Irish moss, panwar gum, mucilage of isapol husks, Veegum®, larch arabogalactan, gelatin, methylcellulose, ethylcellulose, carboxymethylcellulose, hydroxypropylmethylcellulose, polyacrylic acid (e.g., Carbopol), calcium silicate, calcium phosphate, dicalcium phosphate, calcium sulfate, kaolin, sodium chloride, polyethylene glycol; and combinations thereof.

In yet another embodiment, the compositions of the present invention can further comprise a sweetening agent, a flavoring agent, a protecting agent, a plasticizer, a wax, an elastomeric solvent, a filler material, a preservative, or combinations thereof. In still yet another embodiment, the compositions of the present invention can further comprise a lubricating agent, a wetting agent, an emulsifying agent, a solubilizing agent, a suspending agent, a coloring agent, a disintegrating agent, or combinations thereof. In a preferred embodiment, the average particle size of the drug in the compositions described herein is about 20 microns, as compared to a typical average drug particle size of from about 75 to about 100 microns. In another preferred embodiment, the average particle size of the drug in the compositions described herein is less than or equal to the average particle size of the carrier ingredients (e.g., gum base, binders, etc.).

In preferred embodiments of the present invention, the pharmaceutically acceptable salt of pilocarpine is pilocarpine hydrochloride and the binary buffer system comprises sodium carbonate and sodium bicarbonate. In certain instances, the composition comprises from about 0.01 to about 1.0 weight percent pilocarpine hydrochloride; from about 0.1 to about 3.0 weight percent sodium carbonate; and from about 3.0 to about 6.0 weight percent sodium bicarbonate. In a particularly preferred embodiment, the composition comprises from about 0.07 to about 0.2 weight percent pilocarpine hydrochloride; about 0.85 weight percent sodium carbonate; and about 4.5 weight percent sodium bicarbonate. Such compositions are preferably formulated in the form of a chewing gum for buccal administration. As a result, upon mastication of the chewing gum, pilocarpine is delivered across the buccal mucosa. In other preferred embodiments, the weight of the chewing gum is from about 2000 to about 3000 mg. In still other preferred embodiments, the chewing gum is stable upon storage for at least 3 months at 25° C. or 30° C.

In another aspect, the present invention provides a composition for delivery of pilocarpine across the oral mucosa, the composition comprising:
(a) pilocarpine or a pharmaceutically acceptable salt thereof;
(b) a carrier; and
(c) a binary buffer system comprising a strong base and a weak base,
   wherein the amount of the strong base is less than the amount of the weak base.

In one embodiment, the binary buffer system raises the pH of saliva to a pH greater than about 7.5, irrespective of the starting pH of saliva. Preferably, the binary buffer system raises the pH of saliva to a pH of from about 8.0 to about 10.0, irrespective of the starting pH of saliva. Suitable pharmaceutically acceptable salts of pilocarpine are described above.

In another embodiment, the strong base is a carbonate salt. Suitable carbonate salts are described above. In yet another embodiment, the weak base is a bicarbonate salt. Suitable bicarbonate salts are described above. In a particularly preferred embodiment, the strong base is sodium carbonate and the weak base is sodium bicarbonate. Preferred amounts and weight ratios of the strong base to the weak base are described above.

In yet another embodiment, the compositions of the present invention are in any of the dosage forms described above. Preferably, pilocarpine is delivered across an oral mucosa as described above, e.g., a chewing gum composition can be administered buccally so that pilocarpine is delivered across the buccal mucosa. In still yet another embodiment, the carrier is selected from the group consisting of a binder, a gum base, and combinations thereof. Suitable binders and gum bases for use in the compositions of the present invention are described above.

In a further embodiment, the compositions of the present invention can further comprise one or more of the additional agents described above. In preferred embodiments, the average particle size of the drug in the compositions described herein is about 20 microns and/or is less than or equal to the average particle size of the carrier ingredients (e.g., gum base, binders, etc.).

In other preferred embodiments of the present invention, the pharmaceutically acceptable salt of pilocarpine is pilocarpine hydrochloride and the binary buffer system comprises sodium carbonate and sodium bicarbonate. Preferred amounts of each of these components is described above, along with preferred dosage forms, their preferred weight, and their preferred stability.

In yet another aspect, the present invention provides a composition for delivery of pilocarpine across the oral mucosa, the composition comprising:
(a) pilocarpine or a pharmaceutically acceptable salt thereof,
(b) a carrier; and
(c) a binary buffer system comprising a strong base or a weak base and a second buffering agent.

In one embodiment, the binary buffer system raises the pH of saliva to a pH greater than about 7.5, irrespective of the starting pH of saliva. Preferably, the binary buffer system raises the pH of saliva to a pH of from about 8.0 to about 10.0, irrespective of the starting pH of saliva. Suitable pharmaceutically acceptable salts of pilocarpine are described above.

In another embodiment, the strong base is a carbonate salt. Suitable carbonate salts are described above. In yet another embodiment, the weak base is a bicarbonate salt. Suitable bicarbonate salts are described above. In still yet another embodiment, the second buffering agent is selected from the group consisting of a metal oxide, a citrate salt, a phosphate salt, a borate salt, an ascorbate salt, an acetate salt, and alkaline starch. In certain instances, the binary buffer system comprises a carbonate salt and a metal oxide, a citrate salt, a phosphate salt, or a borate salt. In certain other instances, the binary buffer system comprises a bicarbonate salt and a metal oxide, a citrate salt, a phosphate salt, or a borate salt. Preferably, the metal oxide is selected from the group consisting of amorphous magnesium oxide and aluminum oxide.

In yet another embodiment, the compositions of the present invention are in any of the dosage forms described above. Preferably, pilocarpine is delivered across an oral mucosa as described above. In still yet another embodiment, the carrier is selected from the group consisting of a binder, a gum base, and combinations thereof. Suitable binders and gum bases for use in the compositions of the present invention are described above.

In a further embodiment, the compositions of the present invention can further comprise one or more of the additional agents described above. In preferred embodiments, the average particle size of the drug in the compositions described herein is about 20 microns and/or is less than or equal to the average particle size of the carrier ingredients (e.g., gum base, binders, etc.).

In still yet another aspect, the present invention provides a composition for delivery of pilocarpine across the oral mucosa, the composition comprising:
(a) pilocarpine or a pharmaceutically acceptable salt thereof;
(b) a carrier; and
(c) a binary buffer system comprising a metal oxide and a citrate, phosphate, or borate salt.

In one embodiment, the binary buffer system raises the pH of saliva to a pH greater than about 7.5, irrespective of the starting pH of saliva. Preferably, the binary buffer system raises the pH of saliva to a pH of from about 8.0 to about 10.0, irrespective of the starting pH of saliva. Suitable pharmaceutically acceptable salts of pilocarpine are described above.

In another embodiment, the metal oxide is selected from the group consisting of magnesium oxide and aluminum oxide. Preferably, the magnesium oxide is amorphous magnesium oxide. Suitable citrate, phosphate, and borate salts include, without limitation, any salt of citric acid, phosphoric acid, or boric acid known in the art. For example, in some embodiments, the citrate salt is selected from the group consisting of sodium citrate, potassium citrate, calcium citrate, magnesium citrate, and ammonium citrate. In other embodiments, the phosphate salt is selected from the group consisting of monobasic sodium phosphate, dibasic sodium phosphate, monobasic potassium phosphate, dibasic potassium phosphate, monobasic calcium phosphate, dibasic calcium phosphate, monobasic magnesium phosphate, dibasic magnesium phosphate, monobasic ammonium phosphate, and dibasic ammonium phosphate. In yet other embodiments, the borate salt is selected from the group consisting of sodium borate, potassium borate, calcium borate, magnesium borate, and ammonium borate. In certain instances, the binary buffer system comprises a metal oxide and a citrate salt. In certain other instances, the binary buffer system comprises a metal oxide and a phosphate salt. In further instances, the binary buffer system comprises a metal oxide and a borate salt.

In yet another embodiment, the compositions of the present invention are in any of the dosage forms described above. Preferably, pilocarpine is delivered across an oral mucosa as described above. In still yet another embodiment, the carrier is selected from the group consisting of a binder, a gum base, and combinations thereof. Suitable binders and gum bases for use in the compositions of the present invention are described above.

In a further embodiment, the compositions of the present invention can further comprise one or more of the additional agents described above. In preferred embodiments, the average particle size of the drug in the compositions described herein is about 20 microns and/or is less than or equal to the average particle size of the carrier ingredients (e.g., gum base, binders, etc.).

In a further aspect, the present invention provides a composition for delivery of pilocarpine across the oral mucosa, the composition comprising:
(a) pilocarpine or a pharmaceutically acceptable salt thereof;
(b) a carrier; and
(c) a ternary buffer system comprising a strong base, a weak base, and a third buffering agent.

In one embodiment, the binary buffer system raises the pH of saliva to a pH greater than about 7.5, irrespective of the starting pH of saliva. Preferably, the binary buffer system raises the pH of saliva to a pH of from about 8.0 to about 10.0, irrespective of the starting pH of saliva. Suitable pharmaceutically acceptable salts of pilocarpine are described above.

In another embodiment, the strong base is a carbonate salt. Suitable carbonate salts are described above. In yet another embodiment, the weak base is a bicarbonate salt. Suitable bicarbonate salts are described above. In a particularly preferred embodiment, the strong base is sodium carbonate and the weak base is sodium bicarbonate. Preferred amounts and weight ratios of the strong base to the weak base are described above. In another embodiment, the third buffering agent is selected from the group consisting of a metal oxide, a citrate salt, a phosphate salt, a borate salt, an ascorbate salt, an acetate salt, and alkaline starch. In certain instances, the ternary buffer system comprises a carbonate salt, a bicarbonate salt, and a metal oxide. Preferably, the metal oxide is selected from the group consisting of amorphous magnesium oxide and aluminum oxide. In certain other instances, the ternary buffer system comprises a carbonate salt, a bicarbonate salt, and a citrate, phosphate, or borate salt.

In yet another embodiment, the compositions of the present invention are in any of the dosage forms described above. Preferably, pilocarpine is delivered across an oral mucosa as described above. In still yet another embodiment, the carrier is selected from the group consisting of a binder, a gum base, and combinations thereof. Suitable binders and gum bases for use in the compositions of the present invention are described above.

In a further embodiment, the compositions of the present invention can further comprise one or more of the additional agents described above. In preferred embodiments, the average particle size of the drug in the compositions described herein is about 20 microns and/or is less than or equal to the average particle size of the carrier ingredients (e.g., gum base, binders, etc.).

In another aspect, the present invention provides a composition for delivery of pilocarpine across the oral mucosa, the composition comprising:
(a) pilocarpine or a pharmaceutically acceptable salt thereof;
(b) a carrier; and
(c) a buffer system comprising a strong base or a weak base and two or more buffering agents selected from the group consisting of a metal oxide, a citrate salt, a phosphate salt, and a borate salt.

In one embodiment, the binary buffer system raises the pH of saliva to a pH greater than about 7.5, irrespective of the starting pH of saliva. Preferably, the binary buffer system raises the pH of saliva to a pH of from about 8.0 to about 10.0, irrespective of the starting pH of saliva. Suitable pharmaceutically acceptable salts of pilocarpine are described above.

In another embodiment, the strong base is a carbonate salt. Suitable carbonate salts are described above. In yet another embodiment, the weak base is a bicarbonate salt. Suitable bicarbonate salts are described above. In certain instances, the buffer system comprises a carbonate salt or a bicarbonate salt, a metal oxide, and a citrate, phosphate, or borate salt. In certain other instances, the buffer system comprises a carbonate salt or a bicarbonate salt, a citrate salt, and a phosphate salt. In certain instances, the buffer system comprises a carbonate salt or a bicarbonate salt, a citrate salt, and a borate salt. In certain other instances, the buffer system comprises a carbonate salt or a bicarbonate salt, a phosphate salt, and a borate salt. Preferably, the metal oxide is selected from the group consisting of amorphous magnesium oxide and aluminum oxide.

In yet another embodiment, the compositions of the present invention are in any of the dosage forms described above. Preferably, pilocarpine is delivered across an oral mucosa as described above. In still yet another embodiment, the carrier is selected from the group consisting of a binder, a gum base, and combinations thereof. Suitable binders and gum bases for use in the compositions of the present invention are described above.

In a further embodiment, the compositions of the present invention can further comprise one or more of the additional agents described above. In preferred embodiments, the average particle size of the drug in the compositions described herein is about 20 microns and/or is less than or equal to the average particle size of the carrier ingredients (e.g., gum base, binders, etc.).

In yet another aspect, the present invention provides a method for treating dry mouth in a subject in need thereof, the method comprising:
administering to the subject a composition comprising a therapeutically effective amount of pilocarpine or a pharmaceutically acceptable salt thereof; a carrier; and a binary buffer system comprising a strong base and a weak base, wherein the amount of the strong base is less than the amount of the weak base.

In one embodiment, the binary buffer system raises the pH of saliva to a pH greater than about 7.5, irrespective of the starting pH of saliva. Preferably, the binary buffer system raises the pH of saliva to a pH of from about 8.0 to about 10.0, irrespective of the starting pH of saliva. Suitable pharmaceutically acceptable salts of pilocarpine are described above.

In a preferred embodiment, the composition delivers pilocarpine across the oral mucosa such as, for example, the buccal mucosa, the sublingual mucosa, or a combination thereof. The compositions of the present invention can be in any of the dosage forms described above. Preferably, the composition is a chewing gum dosage form that is administered buccally so that pilocarpine is delivered across the buccal mucosa. In another embodiment, the carrier is selected from the group consisting of a binder, a gum base, and combinations thereof. Suitable binders and gum bases for use in the compositions of the present invention are described above. Preferably, the compositions of the present invention are useful for treating dry mouth caused by medical conditions including, without limitation, Sjögren's syndrome, xerostomia, mucositis, and stomatotitis. The compositions of the present invention are also useful for treating dry mouth caused by medication such as decongestants, diuretics, antidepressants, antihypertensives, and antihistamines, or dry mouth caused by medical therapy such as radiotherapy for head and neck cancers.

In another embodiment, the strong base is a carbonate salt. Suitable carbonate salts are described above. In yet another embodiment, the weak base is a bicarbonate salt. Suitable bicarbonate salts are described above. In a particularly preferred embodiment, the strong base is sodium carbonate and the weak base is sodium bicarbonate. Preferred amounts and weight ratios of the strong base to the weak base are described above.

In addition to a binary buffer system comprising a strong base and a weak base, wherein the amount of the strong base is less than the amount of the weak base, other buffer systems are suitable for use in the compositions of the present invention. For example, in an alternative embodiment, the binary buffer system comprises a strong base or a weak base and a second buffering agent such as a metal oxide, a citrate salt, a phosphate salt, a borate salt, an ascorbate salt, an acetate salt, and alkaline starch. In another alternative embodiment, the binary buffer system comprises a metal oxide and a citrate, phosphate, or borate salt. In yet another alternative embodiment, the buffer system is a ternary buffer system comprising a strong base, a weak base, and a third buffering agent such as a metal oxide, a citrate salt, a phosphate salt, a borate salt, an ascorbate salt, an acetate salt, and alkaline starch. In still yet another alternative embodiment, the buffer system comprises a strong base or a weak base and two or more buffering agents selected from the group consisting of a metal oxide, a citrate salt, a phosphate salt, and a borate salt.

In yet another embodiment, the compositions of the present invention can further comprise one or more of the additional agents described above. In preferred embodiments, the average particle size of the drug in the compositions described herein is about 20 microns and/or is less than or equal to the average particle size of the carrier ingredients (e.g., gum base, binders, etc.).

In other preferred embodiments of the present invention, the pharmaceutically acceptable salt of pilocarpine is pilocarpine hydrochloride and the binary buffer system comprises sodium carbonate and sodium bicarbonate. Preferred amounts of each of these components is described above, along with preferred dosage forms, their preferred weight, and their preferred stability. In additional preferred embodiments, the composition increases saliva production by about 400 to about 500 percent within about 30 minutes following administration.

A. Pilocarpine

The compositions of the present invention comprise pilocarpine or a pharmaceutically acceptable salt thereof. Suitable pharmaceutically acceptable salts of pilocarpine include, without limitation, pilocarpine hydrochloride, pilocarpine nitrate, pilocarpine sulfate, pilocarpine acetate, pilocarpine citrate, pilocarpine tartrate, pilocarpine zinc chloride monohydrate, pilocarpine salicylate, a concentrated extract of Pilocarpus leaves, and combinations thereof. Preferably, the pharmaceutically acceptable salt is pilocarpine hydrochloride.

In general, pilocarpine is a basic compound having an ionized form and an un-ionized form. In certain instances, pilocarpine is initially present at least partly in an ionized form. In certain other instances, pilocarpine is initially present in an un-ionized form. As described in more detail below, the buffer system of the compositions described herein helps to convert substantially all of pilocarpine from its ionized form to its un-ionized form. Alternatively, the buffer system helps ensure that pilocarpine, initially in an un-ionized form, remains in an un-ionized form.

As used herein, the term “pilocarpine” includes all pharmaceutically acceptable forms of the drug. For example, pilocarpine can be in a racemic or isomeric mixture, a solid complex bound to an ion exchange resin, or the like. In addition, pilocarpine can be in a solvated form. The term “pilocarpine” is also intended to include all pharmaceutically acceptable salts, derivatives, analogs, and extracts of the drug, as well as combinations thereof. For example, the pharmaceutically acceptable salts of pilocarpine include, without limitation, the acetate, succinate, tartrate, bitartrate, dihydrochloride, salicylate, hemisuccinate, citrate, maleate, hydrochloride, carbamate, sulfate, nitrate, and benzoate salt forms thereof, as well as combinations thereof and the like.

Conversion of the ionized form to the un-ionized form for pilocarpine is related to pH according to the formula: pH=pKa+Log₁₀ (un-ionized concentration/ionized concentration). When the pH is the same as the pKa, equimolar concentrations of the un-ionized form and ionized form exist. For basic compounds such as pilocarpine, when the pH is one unit higher than the pKa, the ratio of the un-ionized form to the ionized form is 91:9. Similarly, when the pH is two units higher than the pKa, the ratio of un-ionized form to the ionized form is 100:1. As noted above, the un-ionized form is lipophilic and, therefore, more capable of passing through mucous membranes such as the oral mucosa than the ionized form, which is lipophobic in nature. Accordingly, increasing the pH of the saliva favors conversion of the ionized form into the un-ionized form for basic compounds such as pilocarpine, and the final pH can be determined by making use of the above formula.

For pilocarpine, the tri-substituted nitrogen in the imidazole group controls the extent of ionization and the degree of lipophilicity in any given medium. Typically, the nitrogen in the imidazole group imparts a pKa of about 6.6 to the molecule at 37° C. Therefore, using the above formula, it can be demonstrated that about 90% conversion to an un-ionized form can be achieved for pilocarpine at a pH of from about 7.6 to about 8.6.

In other embodiments of the present invention, at least one local anesthetic is delivered in combination with pilocarpine or a pharmaceutically acceptable salt thereof. Suitable local anesthetics for use in combination with pilocarpine include, without limitation, ester-based anesthetics such as cocaine, procaine, 2-chloroprocaine, tetracaine, benzocaine, amethocaine, chlorocaine, butamben, and dibucaine; amide-based anesthetics such as lidocaine, prilocaine, mepivacaine, ropivocaine, etidocaine, levobupivacaine, and bupivacaine; ester analogs of aconitine, dyclonine, ketamine, pramoxine, safrole, and salicyl alcohol; and combinations thereof.

B. Buffer Systems

The buffer systems of the compositions described herein are capable of raising the pH of saliva to a pH greater than about 7.5, irrespective of the starting pH of saliva. In this way, the buffer system helps convert substantially all of pilocarpine from its ionized form to its un-ionized form. Alternatively, the buffer system helps ensure that pilocarpine, initially in an un-ionized form, remains in an un-ionized form. Although basic buffering agents are typically used in the buffer systems of the present invention, one skilled in the art will appreciate that acidic agents can also be used to adjust the pH of the buffer system as long as the buffer system as a whole raises the pH of saliva to a pH greater than about 7.5 (e.g., about 8-10).

In one embodiment, the present invention provides binary buffer systems comprising a strong base and a weak base, in which the amount of the strong base is less than the amount of the weak base. The concentration of each buffer system component is tailored such that the final salivary pH is achieved and sustained for a period of time, e.g., for at least about 5 minutes, at least about 10 minutes, at least about 20 minutes, or at least about 60 minutes. This typically involves a sensory and safety trial and error type of procedure of adding various amounts of each buffer system component and then measuring the final pH over time. In this way, selection of an appropriate weight ratio for each buffer system component can be easily determined in just a few trials. For example, the weight ratio of the strong base to the weak base can be at least about 1:3, preferably from about 1:3 to about 1:10, more preferably from about 1:4 to about 1:6, and still more preferably about 1:5.

Suitable buffer system components for use in the present invention include, without limitation, carbonate salts, bicarbonate salts, citrate salts, phosphate salts, borate salts, acetate salts, ascorbate salts, metal oxides, alkaline starch, and combinations thereof. In preferred embodiments, the strong base is a carbonate salt and the weak base is a bicarbonate salt. The carbonate salt is generally selected from sodium carbonate, potassium carbonate, calcium carbonate, ammonium carbonate, and magnesium carbonate. Preferably, the carbonate salt is sodium carbonate or potassium carbonate. Most preferably, the carbonate salt is sodium carbonate. Similarly, the bicarbonate salt is generally selected from sodium bicarbonate, potassium bicarbonate, calcium bicarbonate, ammonium bicarbonate, and magnesium bicarbonate. Preferably, the bicarbonate salt is sodium bicarbonate or potassium bicarbonate. Most preferably, the bicarbonate salt is sodium bicarbonate. In some embodiments, dessicant-coated sodium bicarbonate is preferred. The amount of carbonate salt and bicarbonate salt used in the binary buffer system is an amount that is sufficient to raise salivary pH to a pH of about 7.5 or more, preferably about 8.0 or more, and more preferably from about 8.0 to about 10.0, irrespective of the starting pH. The weight ratio of carbonate salt to bicarbonate salt can be at least about 1:3, preferably from about 1:3 to about 1:10, more preferably from about 1:4 to about 1:6, and still more preferably about 1:5.

In view of the above, the buffer systems of the present invention, in some of the most preferred embodiments, are binary buffer systems containing sodium carbonate and sodium bicarbonate, in which the amount of sodium carbonate is less than the amount of sodium bicarbonate.

Alternatively, in another embodiment, the buffer systems of the present invention are binary buffer systems comprising a strong base or weak base and a second buffering agent. The concentration of each buffer system component is tailored such that the final salivary pH is achieved and sustained for a period of time, e.g., for at least about 2 minutes, at least about 5 minutes, at least about 10 minutes, at least about 20 minutes, or at least about 60 minutes.

In preferred embodiments, the strong base is a carbonate salt and the weak base is a bicarbonate salt. Suitable carbonate salts and bicarbonate salts are described above. The amount of carbonate salt or bicarbonate salt used in the binary buffer system is an amount that is sufficient, when used with the second buffering agent, to raise salivary pH to a pH of about 7.5 or more, preferably about 8.0 or more, and more preferably from about 8.0 to about 10.0, irrespective of the starting pH.

The second buffering agent is generally selected from a metal oxide such as magnesium oxide or aluminum oxide; a citrate salt such as sodium citrate, potassium citrate, calcium citrate, magnesium citrate, and ammonium citrate; a phosphate salt such as monobasic sodium phosphate, dibasic sodium phosphate, monobasic potassium phosphate, dibasic potassium phosphate, monobasic calcium phosphate, dibasic calcium phosphate, monobasic magnesium phosphate, dibasic magnesium phosphate, monobasic ammonium phosphate, and dibasic ammonium phosphate; a borate salt such as sodium borate, potassium borate, calcium borate, magnesium borate, and ammonium borate; an ascorbate salt such as potassium ascorbate or sodium ascorbate; an acetate salt such as potassium acetate or sodium acetate; and alkaline starch. However, one skilled in the art will appreciate that any metal oxide or salt of citric acid, phosphoric acid, boric acid, ascorbic acid, or acetic acid is suitable for use in the buffer systems of the present invention. The amount of the second buffering agent used in the binary buffer system is an amount that is sufficient, when used with the strong base or weak base, to raise salivary pH to a pH of about 7.5 or more, preferably about 8.0 or more, and more preferably from about 8.0 to about 10.0, irrespective of the starting pH. In some embodiments, a metal oxide such as magnesium oxide or aluminum oxide is the preferred second buffering agent. In a particularly preferred embodiment, the metal oxide is amorphous magnesium oxide.

In certain instances, the amount of the second buffering agent in the binary buffer system is greater than or equal to the amount of the strong base or weak base. For example, the weight ratio of the second buffering agent to the strong base or weak base can be from about 1:1 to about 10:1, preferably from about 1:1 to about 5:1, and more preferably from about 1:1 to about 3:1. In certain other instances, the amount of the second buffering agent in the binary buffer system is less than or equal to the amount of the strong base or weak base. For example, the weight ratio of the second buffering agent to the strong base or weak base can be from about 1:1 to about 1:10, preferably from about 1:1 to about 1:5, and more preferably from about 1:1 to about 1:3.

Alternatively, in yet another embodiment, the buffer systems of the present invention are binary buffer systems comprising a metal oxide and a citrate, phosphate, or borate salt. The concentration of each buffer system component is tailored such that the final salivary pH is achieved and sustained for a period of time, e.g., for at least about 2 minutes, at least 5 about minutes, at least about 10 minutes, at least about 20 minutes, or at least about 60 minutes.

The metal oxide is typically magnesium oxide and aluminum oxide. Preferably, the magnesium oxide is amorphous magnesium oxide. Suitable citrate, phosphate, and borate salts include, without limitation, any salt of citric acid, phosphoric acid, or boric acid known in the art such as those described above. In certain instances, the binary buffer system comprises a metal oxide and a citrate salt. In certain other instances, the binary buffer system comprises a metal oxide and a phosphate salt. In further instances, the binary buffer system comprises a metal oxide and a borate salt. The amount of the metal oxide used in the binary buffer system is an amount that is sufficient, when used with the citrate, phosphate, or borate salt, to raise salivary pH to a pH of about 7.5 or more, preferably about 8.0 or more, and more preferably from about 8.0 to about 10.0, irrespective of the starting pH. Similarly, the amount of the citrate, phosphate, or borate salt used in the binary buffer system is an amount that is sufficient, when used with the metal oxide, to raise salivary pH to a pH of about 7.5 or more, preferably about 8.0 or more, and more preferably from about 8.0 to about 10.0, irrespective of the starting pH.

In certain instances, the amount of the metal oxide in the binary buffer system is greater than or equal to the amount of the citrate, phosphate, or borate salt. For example, the weight ratio of the metal oxide to the citrate, phosphate, or borate salt can be from about 1:1 to about 10:1, preferably from about 1:1 to about 5:1, and more preferably from about 1:1 to about 3:1. In certain other instances, the amount of the metal oxide in the binary buffer system is less than or equal to the amount of the citrate, phosphate, or borate salt. For example, the weight ratio of the metal oxide to the citrate, phosphate, or borate salt can be from about 1:1 to about 1:10, preferably from about 1:1 to about 1:5, and more preferably from about 1:1 to about 1:3.

Alternatively, in still yet another embodiment, the buffer systems of the present invention are ternary buffer systems comprising a strong base, a weak base, and a third buffering agent. In certain instances, the amount of the strong base is less than the amount of the weak base. The concentration of each buffer system component is tailored such that the final salivary pH is achieved and sustained for a period of time, e.g., for at least about 2 minutes, at least 5 about minutes, at least about 10 minutes, at least about 20 minutes, or at least about 60 minutes. The procedure described above for determining an appropriate weight ratio for each buffer system component can also be applied to ternary buffer systems.

In preferred embodiments, the strong base is a carbonate salt and the weak base is a bicarbonate salt. Suitable carbonate salts and bicarbonate salts are described above. The amount of carbonate salt and bicarbonate salt used in the ternary buffer system is an amount that is sufficient, when used with the third buffering agent, to raise salivary pH to a pH of about 7.5 or more, preferably about 8.0 or more, and more preferably from about 8.0 to about 10.0, irrespective of the starting pH.

The third buffering agent is generally selected from any metal oxide or salt of citric acid, phosphoric acid, boric acid, ascorbic acid, or acetic acid known in the art such as those described above. The amount of the third buffering agent used in the ternary buffer system is an amount that is sufficient, when used with the remaining components, to raise salivary pH to a pH of about 7.5 or more, preferably about 8.0 or more, and more preferably from about 8.0 to about 10.0, irrespective of the starting pH. In some embodiments, a metal oxide such as magnesium oxide or aluminum oxide is the preferred third buffering agent. In a particularly preferred embodiment, the metal oxide is amorphous magnesium oxide.

In certain instances, the amount of the strong base or weak base in the ternary buffer system is greater than or equal to the amount of the third buffering agent. For example, the weight ratio of the strong base or weak base to the third buffering agent can be from about 1:1 to about 10:1, preferably from about 1:1 to about 5:1, and more preferably from about 1:1 to about 3:1. In certain other instances, the amount of the strong base or weak base in the ternary buffer system is less than or equal to the amount of the third buffering agent. For example, the weight ratio of the strong base or weak base to the third buffering agent can be from about 1:1 to about 1:10, preferably from about 1:1 to about 1:5, and more preferably from about 1:1 to about 1:3.

Alternatively, in a further embodiment, the buffer systems of the present invention are buffer systems comprising a strong base or weak base and two or more buffering agents selected from the group consisting of a metal oxide, a citrate salt, a phosphate salt, and a borate salt. The concentration of each buffer system component is tailored such that the final salivary pH is achieved and sustained for a period of time, e.g., for at least about 2 minutes, at least 5 about minutes, at least about 10 minutes, at least about 20 minutes, or at least about 60 minutes.

In preferred embodiments, the strong base is a carbonate salt and the weak base is a bicarbonate salt. Suitable carbonate salts or bicarbonate salts are described above. The amount of carbonate salt or bicarbonate salt used in the buffer system is an amount that is sufficient, when used with the remaining components, to raise salivary pH to a pH of about 7.5 or more, preferably about 8.0 or more, and more preferably from about 8.0 to about 10.0, irrespective of the starting pH.

The two or more buffering agents are generally selected from citrate salts, phosphate salts, borate salts, acetate salts, ascorbate salts, metal oxides, and alkaline starch such as those described above. The amount of the additional buffering agents used in the buffer system is an amount that is sufficient, when used with the strong base or weak base, to raise salivary pH to a pH of about 7.5 or more, preferably about 8.0 or more, and more preferably from about 8.0 to about 10.0, irrespective of the starting pH.

In one embodiment, the amount of the strong base or weak base is greater than or equal to the amount of the metal oxide or the citrate, phosphate, or borate salt. For example, the weight ratio of the strong base or weak base to the metal oxide or the citrate, phosphate, or borate salt can be from about 1:1 to about 10:1, preferably from about 1:1 to about 5:1, and more preferably from about 1:1 to about 3:1. In another embodiment, the amount of the strong base or weak base in the buffer system is less than or equal to the amount of the metal oxide or the citrate, phosphate, or borate salt. For example, the weight ratio of the strong base or weak base to the metal oxide or the citrate, phosphate, or borate salt can be from about 1:1 to about 1:10, preferably from about 1:1 to about 1:5, and more preferably from about 1:1 to about 1:3.

In certain instances, the buffer system comprises a carbonate salt or a bicarbonate salt, a metal oxide, and a citrate, phosphate, or borate salt. In certain other instances, the buffer system comprises a carbonate salt or a bicarbonate salt, a citrate salt, and a phosphate salt. In certain instances, the buffer system comprises a carbonate salt or a bicarbonate salt, a citrate salt, and a borate salt. In certain other instances, the buffer system comprises a carbonate salt or a bicarbonate salt, a phosphate salt, and a borate salt. Preferably, the metal oxide is amorphous magnesium oxide.

While the foregoing discussion has focused on the ability of the buffer system to alter salivary pH to favor substantial conversion to the un-ionized form of a therapeutic agent, it is conceivable that the buffer system may also have subsidiary beneficial effects on the extent of absorption across the oral mucosa. For example, the buffer system may create a final salivary pH that in turn affects the molecular configuration of the therapeutic agent in a way in which absorption across the oral mucosa is increased. It is to be understood that these subsidiary beneficial effects of the buffer system are within the general scope of the buffer system and compositions herein described.

C. Dosage Forms

The compositions of the present invention may take the form of solid, semi-solid, lyophilized powder, or liquid dosage forms, such as, for example, tablets (e.g., chewable, slow-dissolving, quick-dissolving), pills, capsules, lozenges, gums, powders, solutions, suspensions, emulsions, aerosols, or the like. Preferably, the dosage form is a chewing gum, dissolving tablet, chewable tablet, candy, or lozenge.

While each subject possesses unique factors that may affect the rate and extent of absorption of the therapeutic agents described herein, dosage forms such as chewing gums, chewable tablets, dissolving tablets, or lozenges containing a buffer system described herein offer advantages over the traditional dosage forms for oral administration (i.e., Salagen®). For example, each of these dosage forms avoids hepatic first pass metabolism, degradation within the gastrointestinal tract, and drug loss during absorption. Consequently, the amount of therapeutic agent required per dose is less than that which would be required if formulated, for example, in a pill or tablet for oral administration. Similarly, the bioavailability of the therapeutic agent is increased, thereby reducing the time to onset of therapeutic activity as compared to traditional dosage forms for oral administration (see, Example 3 below).

In addition, the preferred dosage forms of the present invention (e.g., chewing gums, chewable tablets, dissolving tablets, lozenges) containing a buffer system in which the amount of a strong base is less than the amount of a weak base offer advantages over dosage forms for oral mucosal administration that do not contain a buffer system in which the amount of a strong base is less than the amount of a weak base (i.e., chewing gum described in U.S. patent application Ser. No. 10/113,088). Importantly, the dosage forms of the present invention have markedly increased in vitro (i.e., shelf-life) and in vivo (e.g., cud size) stability profiles as compared to similar dosage forms described in, e.g., U.S. patent application Ser. No. 10/113,088. As such, the dosage forms of the present invention are capable of providing exceptional mouth-feel physical properties, texture, and stability, resulting in increased patient compliance.

As used herein, the term “dosage form” refers to physically discrete units suitable as unitary dosages for human subjects and other mammals, each unit containing a predetermined quantity of therapeutic agent calculated to produce the desired onset, tolerability, and therapeutic effects, in association with one or more suitable pharmaceutical excipients such as carriers. Methods for preparing such dosage forms are known or will be apparent to those skilled in the art. For example, in some embodiments, a chewing gum dosage form of the present invention can be prepared according to the procedures set forth in U.S. Pat. No. 4,405,647. In other embodiments, a tablet, lozenge, or candy dosage form of the present invention can be prepared according to the procedures set forth, for example, in Remington: The Science and Practice of Pharmacy, 20^{th} Ed., Lippincott, Williams & Wilkins (2003); Pharmaceutical Dosage Forms, Volume 1: Tablets, 2^{nd} Ed., Marcel Dekker, Inc., New York, N.Y. (1989); and similar publications. The dosage form to be administered will, in any event, contain a quantity of the therapeutic agent in a therapeutically effective amount for relief of the condition being treated when administered in accordance with the teachings of this invention.

As used herein, the term “carrier” refers to a typically inert substance used as a diluent or vehicle for a drug such as a therapeutic agent. The term also encompasses a typically inert substance that imparts cohesive qualities to the composition. Suitable carriers for use in the compositions of the present invention include, without limitation, a solid, semi-solid, or liquid such as a binder or a gum base. Non-limiting examples of binders include sugar alcohols such as mannitol, sorbitol, and xylitol; sugars such as lactose, dextrose, sucrose, glucose, and powdered sugar; natural gums such as acacia gum, xanthan gum, guar gum, tara gum, mesquite gum, fenugreek gum, locust bean gum, ghatti gum, and tragacanth gum; other substances such as inositol, molasses, maltodextrin, starch, cellulose, microcrystalline cellulose, polyvinylpyrrolidone, alginate, extract of Irish moss, panwar gum, mucilage of isapol husks, Veegum®, larch arabogalactan, gelatin, methylcellulose, ethylcellulose, carboxymethylcellulose, hydroxypropylmethylcellulose, polyacrylic acid (e.g., Carbopol), calcium silicate, calcium phosphate, dicalcium phosphate, calcium sulfate, kaolin, sodium chloride, polyethylene glycol; and combinations thereof. These binders can be pre-processed to improve their flowability and taste by methods known in the art such as freeze drying (see, e.g., Fundamentals of Freeze-Drying, Pharm. Biotechnol., 14:281-360 (2002); Lyophililization of Unit Dose Pharmaceutical Dosage Forms, Drug. Dev. Ind. Pharm., 29:595-602 (2003)); solid-solution preparation (see, e.g., U.S. Pat. No. 6,264,987); and lubricant dusting and wet-granulation preparation with a suitable lubricating agent (see, e.g., Remington: The Science and Practice of Pharmacy, supra). For example, Mannogem® and Sorbogem®, sold by SPI Pharma Group (New Castle, Del.), are freeze-dried processed forms of mannitol and sorbitol, respectively. Typically, the compositions of the present invention comprise from about 25% to about 90% by weight of the binder, and preferably from about 50% to about 80%. However, one skilled in the art will appreciate that the compositions of the present invention can be made without any binders, e.g., to produce a highly friable dosage form.

Non-limiting examples of gum bases include materials selected from among the many water-insoluble and saliva-insoluble gum base materials known in the art. For example, in some instances, the gum base comprises at least one hydrophobic polymer and at least one hydrophilic polymer. Non-limiting examples of suitable hydrophobic and hydrophilic polymers for gum bases include both natural and synthetic polymers such as elastomers, rubbers, and combinations thereof. Examples of suitable natural polymers include, without limitation, substances of plant origin such as chicle, jelutong, gutta percha, crown gum, and combinations thereof. Examples of suitable synthetic polymers include elastomers such as butadiene-styrene copolymers, isobutylene and isoprene copolymers (e.g., “butyl rubber”), polyethylene, polyisobutylene, polyvinylester (e.g., polyvinyl acetate and polyvinyl acetate phthalate), and combinations thereof. In other instances, the gum base comprises a mixture of butyl rubber (i.e., isobutylene and isoprene copolymer), polyisobutylene, and optionally, polyvinylacetate (e.g., having a molecular weight of approximately 12,000). The inclusion of a hydrophilic polymer such as polyvinylacetate to a butyl rubber-based gum base can further act synergistically on the absorption of the therapeutic agent. Typically, the gum base comprises from about 25% to about 75% by weight of these polymers, and preferably from about 30% to about 60%.

The compositions of the present invention can additionally include lubricating agents; wetting agents; emulsifying agents; solubilizing agents; suspending agents; preserving agents (i.e., preservatives) such as methyl-, ethyl-, and propyl-hydroxy-benzoates, butylated hydroxytoluene, butylated hydroxyanisole, sodium nitrate, sodium nitrite, sulfites, and disodium EDTA; sweetening agents; flavoring agents; coloring agents; and disintegrating agents (i.e., dissolving agents) such as crospovidone as well as croscarmellose sodium and other cross-linked cellulose polymers.

Lubricating agents can be used to prevent adhesion of the dosage form to the surface of the dies and punches, and to reduce inter-particle friction. Lubricating agents may also facilitate ejection of the dosage form from the die cavity and improve the rate of granulation flow during processing. Examples of suitable lubricating agents include, without limitation, magnesium stearate, calcium stearate, zinc stearate, stearic acid, simethicone, silicon dioxide, talc, hydrogenated vegetable oil, polyethylene glycol, mineral oil, and combinations thereof. The compositions of the present invention can comprise from about 0% to about 10% by weight of the lubricating agent, and preferably from about 1% to about 5%.

Sweetening agents can be used to improve the palatability of the composition by masking any unpleasant tastes it may have. Examples of suitable sweetening agents include, without limitation, compounds selected from the saccharide family such as the mono-, di-, tri-, poly-, and oligosaccharides; sugars such as sucrose, glucose (corn syrup), dextrose, invert sugar, fructose, maltodextrin, and polydextrose; saccharin and salts thereof such as sodium and calcium salts; cyclamic acid and salts thereof, dipeptide sweeteners; chlorinated sugar derivatives such as sucralose and dihydrochalcone; sugar alcohols such as sorbitol, sorbitol syrup, mannitol, xylitol, hexa-resorcinol, and the like, and combinations thereof. Hydrogenated starch hydrolysate, and the potassium, calcium, and sodium salts of 3,6-dihydro-6-methyl-1-1,2,3-oxathiazin-4-one-2,2-dioxide may also be used. Of the foregoing, sorbitol, mannitol, and xylitol, either alone or in combination, are preferred sweetening agents. The compositions of the present invention can comprise from about 0% to about 80% by weight of the sweetening agent, preferably from about 5% to about 75%, and more preferably from about 25% to about 50%.

Flavoring agents can also be used to improve the palatability of the composition. Examples of suitable flavoring agents include, without limitation, natural and/or synthetic (i.e., artificial) compounds such as peppermint, spearmint, wintergreen, cinnamon, menthol, cherry, strawberry, watermelon, grape, banana, peach, pineapple, apricot, pear, raspberry, lemon, grapefruit, orange, plum, apple, fruit punch, passion fruit, chocolate (e.g., white, milk, dark), vanilla, caramel, coffee, hazelnut, combinations thereof, and the like. Coloring agents can be used to color code the composition, for example, to indicate the type and dosage of the therapeutic agent therein. Suitable coloring agents include, without limitation, natural and/or artificial compounds such as FD & C coloring agents, natural juice concentrates, pigments such as titanium oxide, silicon dioxide, and zinc oxide, combinations thereof, and the like. The compositions of the present invention can comprise from about 0% to about 10% by weight of the flavoring and/or coloring agent, preferably from about 0.1% to about 5%, and more preferably from about 2% to about 3%.

1. Chewing Gums

When the dosage form is a chewing gum, the compositions of the present invention comprise pilocarpine or a pharmaceutically acceptable salt thereof, a carrier such as a gum base, a binary buffer system, and optionally a protecting agent. The chewing gum composition may further comprise lubricating agents, wetting agents, emulsifying agents, solubilizing agents, suspending agents, preserving agents, sweetening agents, flavoring agents, and coloring agents. Typically, the chewing gum composition comprises from about 0.001% to about 10.0% by weight of pilocarpine (in whatever chosen form), preferably from about 0.005% to about 2.0%, and more preferably from about 0.01% to about 1.0%. In some embodiments, from about 0.07% to about 0.2% pilocarpine is used. One skilled in the art understands that the foregoing percentages will vary depending upon the particular source of pilocarpine utilized, the amount of pilocarpine desired in the final formulation, as well as on the particular release rate of pilocarpine desired. The buffer system of the inventive pilocarpine chewing gum composition provides for a final salivary pH in excess of at least about 7.5, preferably at least about 8.0, and more preferably from about 8.0 to about 10.0. The chewing gum composition typically comprises from about 20% to about 95% by weight of the gum base, preferably from about 40% to about 90%, and more preferably from about 70% to about 80%.

The chewing gum composition may further comprise a protecting agent. The protecting agent coats at least part of the therapeutic agent, typically upon the mixing of the two agents. The protecting agent may be mixed with the therapeutic agent in a ratio of from about 0.1 to about 100 by weight, preferably in a ratio of from about 1 to about 50, and more preferably in a ratio of about 1 to about 10. Without being bound to any particular theory, the protecting agent reduces the adhesion between the therapeutic agent and the gum base so that the therapeutic agent may be more easily released from the gum base. In this way, the therapeutic agent may be delivered across the mucous membranes of the oral cavity within about 5 to about 20 minutes of chewing, preferably within about 10 minutes of chewing. A variety of different protecting agents may be used. Examples of suitable protecting agents include, without limitation, calcium stearate, glycerin monostearate, glyceryl behenate, glyceryl palmitostearate, hydrogenated castor oil, hydrogenated vegetable oil type I, light mineral oil, magnesium lauryl sulfate, magnesium stearate, mineral oil, poloxamer, polyethylene gycol, sodium benzoate, sodium chloride, sodium lauryl sulfate, stearic acid, cab-o-sil, talc, zinc stearate, and combinations thereof.

The gum base may additionally include plasticizers such as softeners or emulsifiers. Such plasticizers may, for example, help reduce the viscosity of the gum base to a desirable consistency and improve its overall texture and bite. Plasticizers may also facilitate the release of the therapeutic agent upon mastication. Non-limiting examples of plasticizers include lecithin, mono- and diglycerides, lanolin, stearic acid, sodium stearate, potassium stearate, glycerol triacetate, glycerol monostearate, glycerin, and combinations thereof. The gum base typically comprises from about 0% to about 20% by weight of the plasticizer, and more typically from about 5% to about 15%.

The gum base may further comprise waxes such as beeswax and microcrystalline wax, fats or oils such as soybean and cottonseed oil, and combinations thereof. Typically, the gum base comprises from about 0% to about 25% by weight of these waxes and oils, and more typically comprises from about 15% to about 20%.

In addition, the gum base may further comprise one or more elastomeric solvents such as rosins and resins. Non-limiting examples of such solvents include methyl, glycerol, and pentaerythritol esters of rosins, modified rosins such as hydrogenated, dimerized or polymerized rosins, or combinations thereof (e.g., pentaerythritol ester of partially hydrogenated wood rosin, pentaerythritol ester of wood rosin, glycerol ester of wood rosin, glycerol ester of partially dimerized rosin, glycerol ester of polymerized rosin, glycerol ester of tall oil rosin, glycerol ester of wood rosin and partially hydrogenated wood rosin and partially hydrogenated methyl ester of rosin such as polymers of alpha-pinene or beta-pinene, terpene resins including polyterpene, and combinations thereof). Typically, the gum base comprises from about 0% to about 75% of the elastomeric solvent, and more typically less than about 10%.

The gum base may further comprise a filler material to enhance the chewability of the final chewing gum composition. Fillers that are substantially non-reactive with other components of the final chewing gum formulation are preferable. Examples of suitable fillers include, without limitation, calcium carbonate, magnesium silicate (i.e., talc), dicalcium phosphate, metallic mineral salts (e.g., alumina, aluminum hydroxide, and aluminum silicates), and combinations thereof. Typically, the gum base comprises from about 0% to about 30% by weight of the filler, and more typically from about 10% to about 20%.

One skilled in the art will appreciate that the gum base need not be prepared from its individual components. For example, the gum base can be purchased with the desired ingredients contained therein, and can be modified to include additional agents. Several manufacturers produce gum bases suitable for use with the described chewing gum compositions. Examples of such gum bases include, without limitation, Pharmagum™ M, S, or C (SPI Pharma Group; New Castle, Del.). In general, Pharmagum™ comprises a mixture of gum base (e.g., butyl rubber material), sweetening agent, plasticizer, and sugar. Preferably, the gum base is Pharmagum™ M.

In certain instances, the chewing gum composition includes a therapeutic agent centerfill. A centerfill may be particularly suitable when immediate release of the therapeutic agent is preferred. In addition, encapsulating the therapeutic agent in a centerfill may help to mask any undesirable taste that the therapeutic agent may have. In these instances, the gum base surrounds, at least in part, a centerfill. The centerfill comprises at least one therapeutic agent, and may be a liquid or semi-liquid material. The centerfill material can be a synthetic polymer, a semi-synthetic polymer, low-fat, or fat-free and contain one or more sweetening agents, flavoring agents, coloring agents, and/or scenting agents. Preferably, the centerfill includes a buffer system as described herein. Methods for preparing a centerfill chewing gum are described, for example, in U.S. Pat. No. 3,806,290, which is hereby incorporated by reference in its entirety.

The chewing gum compositions can have any desired shape, size, and texture. For example, the chewing gum can have the shape of a stick, tab, gumball, and the like. Similarly, the chewing gum can be any desirable color. For example, the chewing gum can be any shade of red, blue, green, orange, yellow, violet, indigo, and mixtures thereof, and can be color coded to indicate the type and dosage of the therapeutic agent therein. The chewing gum can be individually wrapped or grouped together in pieces for packaging by methods well known in the art.

2. Tablets

When the dosage form is a tablet such as a dissolving tablet or chewable tablet, the compositions of the present invention comprise pilocarpine or a pharmaceutically acceptable salt thereof, a carrier such as a binder, and a binary buffer system. The tablet composition may further comprise lubricating agents, wetting agents, emulsifying agents, solubilizing agents, suspending agents, preserving agents, sweetening agents, flavoring agents, coloring agents, and disintegrating agents. Typically, the tablet compositions of the present invention comprise from about 0.001% to about 10.0% by weight of pilocarpine (in whatever chosen form), and preferably from about 0.01% to about 5.0%. One skilled in the art understands that the foregoing percentages will vary depending upon the particular source of pilocarpine utilized, the amount of pilocarpine desired in the final formulation, as well as on the particular release rate of pilocarpine desired. The buffer system of the tablet composition provides for a final salivary pH in excess of at least about 7.5, preferably at least about 8.0, and more preferably from about 8.0 to about 10.0.

In certain embodiments, the tablet is a dissolving tablet such as a slow-dissolving or quick-dissolving tablet that is dissolved by a subject's saliva, without the need for chewing. For example, a dissolving tablet placed on the subject's tongue can be used for buccal delivery of the therapeutic agent. Alternatively, a dissolving tablet placed underneath the subject's tongue can be used for sublingual delivery of the therapeutic agent. This type of dosage form may be particularly desirable for pediatric and geriatric patients, since small children and aged individuals often have difficulty chewing certain items. Typically, the dissolving tablet is formulated to dissolve within about 1 to about 15 minutes, preferably within about 2 to about 10 minutes, e.g., within about 2, 3, 4, 5, 6, 7, 8, 9, or 10 minutes, following administration. One skilled in the art will understand that quick-dissolving tablets dissolve faster than slow-dissolving tablets, which are typically dissolved gradually rather than rapidly by a subject's saliva.

In certain other embodiments, the tablet is a chewable tablet that is chewed by a subject and formulated to dissolve either rapidly or gradually. For example, a chewable tablet placed on the subject's tongue can be used for buccal delivery of the therapeutic agent. During chewing, the chewable tablet can be moved around within the mouth and can sometimes be parked between the gums and the cheeks or underneath the tongue. As a result, at least a portion of the therapeutic agent contained within a chewable tablet may also be delivered sublingually (i.e., across the sublingual mucosa). Typically, the chewable tablet is formulated to dissolve within about 1 to about 15 minutes, preferably within about 2 to about 10 minutes, e.g., within about 2, 3, 4, 5, 6, 7, 8, 9, or 10 minutes, following administration.

As described above, the dissolving and chewable tablets of the present invention are typically formulated to dissolve within about 1 to 15 minutes following administration. However, while these time frames are amenable to maximum exposure of the therapeutic agent to the oral mucosa (e.g., to the sublingual and/or buccal mucosa), they are not always amenable to user compliance (e.g., users may swallow too frequently and, therefore, hinder maximal transmucosal absorption). Consequently, in certain instances, it may be desirable to strike a balance between patient compliance and maximum exposure time of the therapeutic agent to the oral mucosa. This can be accomplished, for example, by reducing the tablet size (e.g., from about 700-800 mg to about 200-300 mg) without reducing the concentration or amount per unit dose of the buffer system or the therapeutic agent. In addition, subtle changes to the tablet formulation such as, for example, replacing one flavoring agent for another (e.g., chocolate for spearmint) or replacing one binder or sweetening agent for another (e.g., lactose for mannitol or sorbitol) may be used to reduce salivation.

The carrier present in the tablets of the present invention is typically a binder that is useful in keeping the tablet in a semi-solid state, and may be a solid or a liquid, and may for example be a high-melting point fat or waxy material. Materials suitable as binders are discussed in detail above and may be used alone or in combination in the tablet compositions of the present invention. In addition, binders such as mannitol, sorbitol, lactose, sucrose, and inositol can impart properties to the tablet that permit or enhance its disintegration in the mouth.

The tablet composition may further comprise a protecting agent. The protecting agent coats at least part of the therapeutic agent, typically upon the mixing of the two agents. The protecting agent may be mixed with the therapeutic agent in a ratio of from about 0.1 to about 100 by weight, preferably in a ratio of from about 1 to about 50, and more preferably in a ratio of about 1 to about 10. Without being bound to any particular theory, the protecting agent reduces the adhesion between the therapeutic agent and the binder so that the therapeutic agent may be more easily released from the binder. In this way, the therapeutic agent may be delivered across the mucous membranes of the oral cavity within about 5 to about 20 minutes, preferably within about 10 minutes. Materials suitable as protecting agents are discussed in detail above and may be used alone or in combination in the tablet compositions of the present invention.

The tablet composition may also comprise one or more elastomeric solvents such as rosins and resins. Non-limiting examples of such solvents are discussed in detail above and may be used alone or in combination in the tablet compositions of the present invention. In addition, the tablet composition may further comprise waxes such as beeswax and microcrystalline wax, fats or oils such as soybean and cottonseed oil, and combinations thereof. Moreover, the tablet composition may additionally include plasticizers such as softeners or emulsifiers. Such plasticizers may, for example, help reduce the viscosity of the salivary solution of the dissolved tablet to a desirable consistency and improve its overall texture and bite and help facilitate the release of the therapeutic agent. Non-limiting examples of such plasticizers are discussed in detail above and may be used alone or in combination in the tablet compositions of the present invention.

In certain instances, the tablet composition includes a therapeutic agent centerfill. A centerfill may be particularly suitable when immediate release of the therapeutic agent is preferred. In addition, encapsulating the therapeutic agent in a centerfill may help to mask any undesirable taste that the therapeutic agent may have. In these instances, the binder surrounds, at least in part, a centerfill. The centerfill comprises at least one therapeutic agent, and may be a liquid or semi-liquid material. The centerfill material can be low-fat or fat free and contain one or more sweetening agents, flavoring agents, coloring agents, and/or scenting agents. Preferably, the centerfill includes a buffer system as described herein.

In certain other instances, the tablet composition of the present invention is multilayered. In this way, the dissolving or chewable tablet can be designed to provide more than one therapeutic agent, e.g., pilocarpine or a pharmaceutically acceptable salt thereof in combination with one or more local anesthetics. For example, with a bi-layered tablet, the first layer can contain pilocarpine and the second layer can contain one or more local anesthetics. Typically, the first layer comprises the dissolving or chewable portion of the tablet, and the second (i.e., subsequent) layer is coated by the first layer. This type of formulation may be particularly suitable when immediate release of pilocarpine, followed by gastrointestinal absorption of a second therapeutic agent, is desirable. Gastrointestinal absorption of the second therapeutic agent may be desirable, for example, in order to mitigate co-morbid symptoms or to sustain the therapeutic benefit of pilocarpine in the dissolving or the chewable portion of the tablet. Alternatively, the second layer is present as a layer lateral to the first layer. The second layer typically comprises at least one therapeutic agent, and can also comprise one or more sweetening agents, flavoring agents, coloring agents, and scenting agents as described above. In some instances, the second layer further includes buffer system as described herein.

In still other instances, the combination of pilocarpine or a pharmaceutically acceptable salt thereof with one or more additional therapeutic agents need not take the form of a multilayered tablet, but instead comprises a single homogenous tablet layer. This type of formulation may also be used in the case where gastrointestinal absorption of at least one therapeutic agent is desirable. In this case, the relative extent of ionization of the two or more therapeutic agents determines how they are to be absorbed. For example, those therapeutic agents that are un-ionized are absorbed through the oral mucosa, while the ionized agents are swallowed for gastrointestinal absorption.

The tablet compositions can have any desired shape, size, and texture. For example, the tablet can have the shape of a stick, tab, pellet, sphere, and the like. Similarly, the tablet can be any desirable color. For example, the tablet can be any shade of red, blue, green, orange, yellow, violet, indigo, and mixtures thereof, and can be color coded to indicate the type and dosage of the therapeutic agent therein. The tablets can be individually wrapped or grouped together in pieces for packaging by methods well known in the art.

3. Lozenges

When the dosage form is a lozenge or candy, the compositions of the present invention comprise pilocarpine or a pharmaceutically acceptable salt thereof, a carrier such as a binder, and a binary buffer system. The lozenge or candy composition may further comprise lubricating agents, wetting agents, emulsifying agents, solubilizing agents, suspending agents, preserving agents, sweetening agents, flavoring agents, coloring agents, and disintegrating agents. A general discussion of lozenges and candies is provided, e.g., in Pharmaceutical Dosage Forms, Volume 1: Tablets, 2^{nd} Ed., Marcel Dekker, Inc., New York, N.Y., pages 75-418 (1989). Typically, the lozenge compositions of the present invention comprise from about 0.001% to about 10.0% by weight of pilocarpine (in whatever chosen form), and more preferably from about 0.01% to about 5.0%. One skilled in the art understands that the foregoing percentages will vary depending upon the particular source of pilocarpine utilized, the amount of pilocarpine desired in the final formulation, as well as on the particular release rate of pilocarpine desired. The buffer system of the lozenge composition provides for a final salivary pH in excess of at least about 7.5, preferably at least about 8.0, and more preferably from about 8.0 to about 10.0.

In certain embodiments, the lozenge or candy is dissolved by a subject's saliva, without the need for chewing. For example, a lozenge placed on the subject's tongue can be used for buccal delivery of the therapeutic agent. Alternatively, a lozenge placed underneath the subject's tongue can be used for sublingual delivery of the therapeutic agent. This type of dosage form may be particularly desirable for pediatric and geriatric patients, since small children and aged individuals often have difficulty chewing certain items. Typically, the lozenge is formulated to dissolve within about 1 to about 15 minutes, preferably within about 2 to about 10 minutes, e.g., within about 2, 3, 4, 5, 6, 7, 8, 9, or 10 minutes, following administration.

As described above, the lozenges the present invention are typically formulated to dissolve within about 1 to 15 minutes following administration. However, while these time frames are amenable to maximum exposure of the therapeutic agent to the oral mucosa (e.g., to the sublingual and/or buccal mucosa), they are not always amenable to user compliance (e.g., users may swallow too frequently and, therefore, hinder maximal transmucosal absorption). Consequently, in certain instances, it may be desirable to strike a balance between patient compliance and maximum exposure time of the therapeutic agent to the oral mucosa. This can be accomplished, for example, by reducing the lozenge size (e.g., from about 700-800 mg to about 200-300 mg) without reducing the concentration or amount per unit dose of the buffer system or the therapeutic agent. In addition, subtle changes to the lozenge formulation such as, for example, replacing one flavoring agent for another (e.g., chocolate for spearmint) or replacing one binder or sweetening agent for another (e.g., lactose for mannitol or sorbitol) may be used to reduce salivation.

The carrier present in the lozenges of the present invention is typically a binder that is useful in keeping the lozenge in a semi-solid state, and may be a solid or a liquid, and may for example be a high-melting point fat or waxy material. Materials suitable as binders are discussed in detail above and may be used alone or in combination in the lozenge compositions of the present invention. In addition, binders such as mannitol, sorbitol, lactose, sucrose, and inositol can impart properties to the lozenge that permit or enhance its disintegration in the mouth.

The lozenge composition may further comprise a protecting agent. The protecting agent coats at least part of the therapeutic agent, typically upon the mixing of the two agents. The protecting agent may be mixed with the therapeutic agent in a ratio of from about 0.1 to about 100 by weight, preferably in a ratio of from about 1 to about 50, and more preferably in a ratio of about 1 to about 10. Without being bound to any particular theory, the protecting agent reduces the adhesion between the therapeutic agent and the binder so that the therapeutic agent may be more easily released from the binder. In this way, the therapeutic agent may be delivered across the mucous membranes of the oral cavity within about 5 to about 20 minutes, preferably within about 10 minutes. Materials suitable as protecting agents are discussed in detail above and may be used alone or in combination in the lozenge compositions of the present invention.

The lozenge composition may also comprise one or more elastomeric solvents such as rosins and resins. Non-limiting examples of such solvents are discussed in detail above and may be used alone or in combination in the tablet compositions of the present invention. In addition, the lozenge composition may further comprise waxes such as beeswax and microcrystalline wax, fats or oils such as soybean and cottonseed oil, and combinations thereof. Moreover, the lozenge composition may additionally include plasticizers such as softeners or emulsifiers. Such plasticizers may, for example, help reduce the viscosity of the salivary solution of the dissolved lozenge to a desirable consistency and improve its overall texture and bite and help facilitate the release of the therapeutic agent. Non-limiting examples of such plasticizers are discussed in detail above and may be used alone or in combination in the lozenge compositions of the present invention.

In certain instances, the lozenge composition includes a therapeutic agent centerfill. A centerfill may be particularly suitable when immediate release of the therapeutic agent is preferred. In addition, encapsulating the therapeutic agent in a centerfill may help to mask any undesirable taste that the therapeutic agent may have. In these instances, the binder surrounds, at least in part, a centerfill. The centerfill comprises at least one therapeutic agent, and may be a liquid or semi-liquid material. The centerfill material can be low-fat or fat free and contain one or more sweetening agents, flavoring agents, coloring agents, and/or scenting agents. Preferably, the centerfill includes a buffer system as described herein.

In certain other instances, the lozenge composition of the present invention is multilayered. In this way, the lozenge can be designed to provide more than one therapeutic agent, e.g., pilocarpine or a pharmaceutically acceptable salt thereof in combination with one or more local anesthetics. For example, with a bi-layered lozenge, the first layer can contain pilocarpine and the second layer can contain one or more local anesthetics. Typically, the first layer comprises the dissolving portion of the lozenge, and the second (i.e., subsequent) layer is coated by the first layer. This type of formulation may be particularly suitable when immediate release of pilocarpine, followed by gastrointestinal absorption of a second therapeutic agent, is desirable. Gastrointestinal absorption of the second therapeutic agent may be desirable, for example, in order to mitigate co-morbid symptoms or to sustain the therapeutic benefit of pilocarpine in the dissolving portion of the lozenge. Alternatively, the second layer is present as a layer lateral to the first layer. The second layer typically comprises at least one therapeutic agent, and can also comprise one or more sweetening agents, flavoring agents, coloring agents, and scenting agents as described above. In some instances, the second layer further includes a buffer system as described herein.

In still other instances, the combination of pilocarpine or a pharmaceutically acceptable salt thereof with one or more additional therapeutic agents need not take the form of a multilayered lozenge, but instead comprises a single homogenous lozenge layer. This type of formulation may also be used in the case where gastrointestinal absorption of at least one therapeutic agent is desirable. In this case, the relative extent of ionization of the two or more therapeutic agents determines how they are to be absorbed. For example, those therapeutic agents that are un-ionized are absorbed through the oral mucosa, while the ionized agents are swallowed for gastrointestinal absorption.

The lozenge compositions can have any desired shape, size, and texture. For example, the lozenge can have the shape of a stick, tab, pellet, sphere, and the like. Similarly, the lozenge can be any desirable color. For example, the lozenge can be any shade of red, blue, green, orange, yellow, violet, indigo, and mixtures thereof, and can be color coded to indicate the type and dosage of the therapeutic agent therein. The lozenges can be individually wrapped or grouped together in pieces for packaging by methods well known in the art.

D. Methods of Administration

The compositions of the present invention are useful in therapeutic applications, e.g., for treating dry mouth. Importantly, the compositions of the present invention provide the rapid delivery of pilocarpine across the oral mucosa by raising the pH of saliva to a pH greater than about 7.5, irrespective of the starting pH of saliva. In particular, the delivery of the therapeutic agent across the oral mucosa avoids hepatic first pass metabolism, degradation within the gastrointestinal tract, and drug loss during absorption. As a result, the therapeutic agent reaches the systemic circulation in a substantially shorter period of time and at a substantially higher concentration than with traditional oral (e.g., tablet) administration.

The compositions of the present invention have particular utility in the area of human and veterinary therapeutics. Generally, administered dosages will be effective to deliver picomolar to micromolar concentrations of pilocarpine to the appropriate site.

Administration of the compositions of the present invention is preferably carried out via any of the accepted modes of administration to the mucous membranes of the oral cavity. Examples of suitable sites of administration within the oral mucosa include, without limitation, the mucous membranes of the floor of the mouth (sublingual mucosa), the cheeks (buccal mucosa), the gums (gingival mucosa), the roof of the mouth (palatal mucosa), the lining of the lips, and combinations thereof. These regions differ from each other with respect to their anatomy, drug permeability, and physiological response to drugs. Preferably, the compositions of the present invention are administered to the buccal mucosa, sublingual mucosa, or a combination thereof.

The oral mucosa, possessing a rich blood supply and suitable drug permeability, is an especially attractive route of administration for systemic drug delivery. Furthermore, delivery of a therapeutic agent across the oral mucosa bypasses hepatic first pass metabolism, avoids enzymatic degradation within the gastrointestinal tract, and provides a more suitable enzymatic flora for drug absorption. As used herein, the term “buccal delivery” refers to the administration of a therapeutic agent across the mucous membranes lining the cheeks. The term “sublingual delivery” as used herein refers to the administration of a therapeutic agent across the mucous membranes lining the floor of the mouth and/or the ventral tongue.

The oral mucosa is composed of an outermost layer of stratified squamous epithelium. Beneath this layer lies a basement membrane, i.e., the lamina propria, followed by the submucosa as the innermost layer. The epithelium of the oral mucosa is similar to the stratified squamous epithelia found in the rest of the body in that it contains a mitotically active basal cell layer, advancing through a number of differentiating intermediate layers to the superficial layers, where cells are shed from the surface of the epithelium (Gandhi et al., Ind. J. Pharm. Sci., 50:145-152 (1988)). For example, the epithelium of the buccal mucosa is about 40-50 cell layers thick, while that of the sublingual epithelium contains somewhat fewer cell layers. The epithelial cells increase in size and become flatter as they travel from the basal layers to the superficial layers.

The turnover time for buccal mucosal epithelium, estimated at 5-6 days, is representative of the turnover time for sublingual mucosal epithelium as well as other epithelia in the oral mucosa (Harris et al., J. Pharm. Sci., 81:1-10 (1992)). The thickness of the oral mucosa varies depending on the site in the oral cavity. For example, the buccal mucosa measures at about 500-800 μm in thickness, while the hard and soft palatal mucosa, the sublingual mucosa, the ventral tongue, and the gingival mucosa measure at about 100-200 μm in thickness. The composition of the epithelium also varies depending on the site in the oral cavity. For example, the mucosae of areas subject to mechanical stress (i.e., the gingivae and hard palate) are keratinized similar to the epidermis. However, the mucosae of the soft palate, the sublingual region, and the buccal region are not keratinized (Harris et al., supra). The keratinized epithelia contain neutral lipids like ceramides and acylceramides, which have been associated with providing a barrier function. As a result, these epithelia are relatively impermeable to water. In contrast, non-keratinized epithelia, such as sublingual and buccal epithelia, do not contain acylceramides and have only small amounts of ceramide (Wertz et al., Crit. Rev. Ther. Drug Carr. Sys., 8:237-269 (1991); Squier et al., J. Invest. Dermat., 96:123-126 (1991); Squier et al., in Oral Mucosal Drug Delivery, Ed. M. J. Rathbone, Marcel Dekker, Inc., New York, N.Y., 1-26 (1996)). Non-keratinized epithelia also contain small amounts of neutral but polar lipids, e.g., cholesterol sulfate and glucosyl ceramides. As such, these epithelia have been found to be considerably more permeable to water than keratinized epithelia (Harris et al., supra; Wertz et al., supra; Squier et al., supra, 1991).

In general, the oral mucosa is a somewhat leaky epithelia intermediate between that of the epidermis and intestinal mucosa. For example, the permeability of the buccal mucosa is estimated to be about 4-4000 times greater than that of skin (Galey et al., J. Invest. Dermat., 67:713-717 (1976)). The permeability of different regions of the oral mucosa generally decrease in the order of sublingual mucosa greater than buccal mucosa, and buccal mucosa greater than palatal mucosa (Harris et al., supra). This permeability is generally based upon the relative thickness and degree of keratinization of these membranes, with the sublingual mucosa being relatively thin and non-keratinized, the buccal mucosa being thicker and non-keratinized, and the palatal mucosa being intermediate in thickness, but keratinized.

The epithelial cells of the oral mucosa are surrounded by mucus comprising primarily complexes of proteins and carbohydrates that may or may not be attached to certain regions on the cell surface. The mucus may play a role in cell-cell adhesion, as well as acting as a lubricant, allowing cells to move relative to one another (Tabak et al., J. Oral Pathol., 11:1-17 (1982)). In stratified squamous epithelia found elsewhere in the body, mucus is synthesized by specialized mucus secreting cells such as goblet cells; however, in the oral mucosa, mucus is secreted by the major and minor salivary glands as part of saliva (Tabak et al., supra; Rathbone et al., Adv. Drug Del. Rev., 13:1-22 (1994)). At physiological pH, the mucus network carries a negative charge due to the sialic acid and sulfate residues present on the carbohydrates. At this pH, mucus can form a strongly cohesive gel structure that binds to the epithelial cell surface as a gelatinous layer (Gandhi et al., supra). Without being bound to any particular theory, the buffer systems of the present invention neutralize the sialic acid residues present on the carbohydrates and prevent them from interacting with the therapeutic agent, thereby further enhancing drug permeation.

Another feature of the environment of the oral cavity is the presence of saliva produced by the salivary glands. Saliva is the protective fluid for all tissues of the oral cavity. Saliva is an aqueous fluid with about 1% organic and inorganic materials. The major determinant of the salivary composition is the flow rate, which in turn depends upon factors such as the time of day, the type of stimulus, and the degree of stimulation. The salivary pH typically ranges from about 5.5 to about 7.0, depending on the flow rate. For example, at high flow rates, the sodium and bicarbonate concentrations increase, leading to an increase in the pH. Because the daily salivary volume is between about 0.5 to about 2 liters, the oral cavity provides an aqueous environment for the hydration and/or dissolution of the oral mucosal dosage forms of the present invention.

The sublingual mucosa is the most highly permeable region of the oral cavity, and provides rapid absorption and high bioavailability of a drug in a convenient, accessible, and well-accepted route of administration (Harris et al., supra). Suitable sublingual dosage forms include, without limitation, tablets (e.g., quick-dissolving, slow-dissolving), lozenges, candy, and soft gelatin capsules filled with liquid drug. Such systems create a very high drug concentration in the sublingual region before they are systemically absorbed across the sublingual mucosa. Although the buccal mucosa is considerably less permeable than the sublingual area, rapid absorption and high bioavailability of a drug can also be observed with buccal administration. Suitable buccal dosage forms include, without limitation, chewing gums, tablets (e.g., quick-dissolving, slow-dissolving), lozenges, candy, and the like. Both the buccal mucosa and the sublingual mucosa are far superior to the gastrointestinal tract for providing increased absorption and bioavailability of a drug.

To increase the permeability of drugs through the oral mucosa, penetration enhancers can be included in the dosage forms of the present invention. The penetration enhancers may be of the type that alters the nature of the oral mucosa to enhance penetration, or of the type that alters the nature of the therapeutic agent to enhance penetration through the oral mucosa. Suitable penetration enhancers include, without limitation, polyoxyethylene 23-lauryl ether, aprotin, azone, benzalkonium chloride, cetylpyridinium chloride, cetyltrimethylammonium bromide, cyclodextrin, dextran sulfate, lauric acid, propylene glycol, lysophosphatidylcholine, menthol, methoxysalicylate, methyloleate, oleic acid, phosphatidylcholine, polyoxyethylene, polysorbate 80, sodium ethylenediaminetetraacetic acid (“EDTA”), sodium deoxycholate, sodium glycocholate, sodium glycodeoxycholate, sodium lauryl suflate, sodium salicylate, sodium taurocholate, sodium taurodeoxycholate, as well as certain sulfoxides and glycosides, and combinations thereof.

### IV. EXAMPLES

The following examples are offered to illustrate, but not to limit, the claimed invention.

### Example 1

### Pilocarpine Chewing Gum Stability Studies

This example illustrates a comparison of the stability between the inventive pilocarpine chewing gum compositions and the pilocarpine chewing gum composition described in U.S. patent application Ser. No. 10/113,088.

TABLE 1
Comparison of Pilocarpine Chewing Gum Formulations.

| | U.S. Patent Application No. | | Inventive Chewing | |
|---|---|---|---|---|
| Ingredient | 10/113,088 Chewing Gum | | Gum | |
| Pilocarpine | 5 | mg | 2.0, 3.5, or 5.0 | mg |
| Hydrochloride | | | | |
| Sodium Carbonate | 15 | mg | 22 | mg |
| Sodium Bicarbonate | 7.5 | mg | 114 | mg |

Table 2 below shows the 3-month stability data at 25° C. or 30° C. for the pilocarpine chewing gum composition described in U.S. patent application Ser. No. 10/113,088. Tables 3-5 below show the 3-month stability data at 25° C. or 30° C. for the inventive pilocarpine chewing gum compositions containing either 2.0, 3.5, or 5.0 mg pilocarpine hydrochloride. 3 months of stability at 30° C. corresponds to 6 months of shelf-life at 25° C. (room temperature).

TABLE 2
Stability Data for the Pilocarpine Chewing Gum of
U.S. Patent Application No. 10/113,088.

| | | |
|---|---|---|
| Test | Purity of Pilocarpine (%) | Appearance |

AT 25° C.

| | | |
|---|---|---|
| Specification | 90-110 | 0.750″ tan-mottled, round, flat |
| | | bevel-edged uncoated gum tablet |

RESULTS

| | | |
|---|---|---|
| Initial | 95.3 | No changes |
| 1 Month | 95.7 | Soft mushy tablet |
| 2 Month | 89.7 | Soft creamy tablet |
| | (Failed) | |
| 3 Month | Discarded | |

AT 30° C.

| | | |
|---|---|---|
| Specification | 90-110 | 0.750″ tan-mottled, round, flat |
| | | bevel-edged uncoated gum tablet |

RESULTS

| | | |
|---|---|---|
| Initial | 95.3 | No changes |
| 1 Month | Not Analyzed | Semi-solid translucent mass |
| 2 Month | Discarded | |

TABLE 3
Stability Data for the Inventive Pilocarpine Chewing Gum (2 mg).

| | | |
|---|---|---|
| Test | Purity of Pilocarpine (%) | Appearance |

AT 25° C.

| | | |
|---|---|---|
| Specification | 90-110 | 0.750″ tan-mottled, round, flat |
| | | bevel-edged uncoated gum tablet |

RESULTS

| | | |
|---|---|---|
| Initial | 96.0 | No changes |
| 1 Month | 101.0 | No changes |
| 2 Month | 95.0 | No changes |
| 3 Month | 96.0 | No changes |

AT 30° C.

| | | |
|---|---|---|
| Specification | 90-110 | 0.750″ tan-mottled, round, flat |
| | | bevel-edged uncoated gum tablet |

RESULTS

| | | |
|---|---|---|
| Initial | 96.0 | No changes |
| 1 Month | 101.0 | No changes |
| 2 Month | 94.5 | No changes |
| 3 Month | 98.0 | No changes |

TABLE 4
Stability Data for the Inventive Pilocarpine Chewing Gum (3.5 mg).

| | | |
|---|---|---|
| Test | Purity of Pilocarpine (%) | Appearance |

AT 25° C.

| | | |
|---|---|---|
| Specification | 90-110 | 0.750″ tan-mottled, round, flat |
| | | bevel-edged uncoated gum tablet |

RESULTS

| | | |
|---|---|---|
| Initial | 98.3 | No changes |
| 1 Month | 97.1 | No changes |
| 2 Month | 104.3 | No changes |
| 3 Month | 104.9 | No changes |

AT 30° C.

| | | |
|---|---|---|
| Specification | 90-110 | 0.750″ tan-mottled, round, flat |
| | | bevel-edged uncoated gum tablet |

RESULTS

| | | |
|---|---|---|
| Initial | 98.3 | No changes |
| 1 Month | 93.4 | No changes |
| 2 Month | 95.7 | No changes |
| 3 Month | 101.7 | No changes |

TABLE 5
Stability Data for the Inventive Pilocarpine Chewing Gum (5.0 mg).

| | | |
|---|---|---|
| Test | Purity of Pilocarpine (%) | Appearance |

AT 25° C.

| | | |
|---|---|---|
| Specification | 90-110 | 0.750″ tan-mottled, round, flat |
| | | bevel-edged uncoated gum tablet |

RESULTS

| | | |
|---|---|---|
| Initial | 98.8 | No changes |
| 1 Month | 105.6 | No changes |
| 2 Month | 99.4 | No changes |
| 3 Month | 107.0 | No changes |

AT 30° C.

| | | |
|---|---|---|
| Specification | 90-110 | 0.750″ tan-mottled, round, flat |
| | | bevel-edged uncoated gum tablet |

RESULTS

| | | |
|---|---|---|
| Initial | 98.8 | No changes |
| 1 Month | 105.4 | No changes |
| 2 Month | 102.2 | No changes |
| 3 Month | 103.0 | No changes |

As shown in Table 2, the pilocarpine chewing gum composition described in U.S. patent application Ser. No. 10/113,088, in which the amount of sodium carbonate (strong base) is greater than the amount of sodium bicarbonate (weak base), began to decompose within 1 month after storing at room temperature (25° C.). The consistency of the chewing gum was soft and mushy. After storage for another month at room temperature, the chewing gum had a creamy consistency and the purity of pilocarpine had dropped to 89.7%. The chewing gum was discarded after 3 months of storage at room temperature. The decomposition process was accelerated when the tablet was stored at an elevated temperature (30° C.), as the chewing gum was discarded after only 2 months of storage.

However, as shown in Tables 3-5, the inventive pilocarpine chewing gum compositions, in which the amount of sodium carbonate (strong base) is less than the amount of sodium bicarbonate (weak base), remains stable upon storage at both temperatures. In particular, there was no sign of decomposition or any changes to the physical appearance of the inventive pilocarpine chewing gum compositions. The remarkable increase in stability observed for the inventive pilocarpine chewing gum compositions having, e.g., about a 1:5 sodium carbonate:sodium bicarbonate ratio as compared to the pilocarpine chewing gum composition described in U.S. patent application Ser. No. 10/113,088 having a 2:1 sodium carbonate:sodium bicarbonate ratio simply could not have been predicted based on any information available in the prior art.

Additionally, it was observed that the pilocarpine chewing gum composition described in U.S. patent application Ser. No. 10/113,088 exhibited poor mouth-feel properties. For example, the chewing gum composition was difficult to masticate as the composition liquefied and left little to no cud after chewing. In contrast, the inventive pilocarpine chewing gum compositions did not liquefy and left a large cud after chewing. Thus, the inventive pilocarpine chewing gum compositions provide a substantially better mouth-feel experience and chewing texture.

### Example 2

### Pilocarpine Chewing Gum Compositions

This example illustrates the pilocarpine chewing gum compositions of the present invention.

Pilocarpine can be formulated as a chewing gum composition as described above. In these embodiments, the unit dose or serving of the chewing gum comprises from about 0.1 to about 100 milligrams (mg) pilocarpine, preferably from about 1 to about 50 mg, and more preferably from about 2 to about 25 mg. In preferred embodiments, the unit dose comprises from about 2 to about 5 mg pilocarpine, e.g., about 2.0, 2.5, 3.0, 3.5, 4.0, 4.5, 5.0. Extra pilocarpine, for example, up to from about 10% to about 25% by weight, can be added as “overage” or as the amount that may be expected to be “washed away” and not otherwise released or absorbed during mastication.

Given in weight percentages, the inventive pilocarpine chewing gum composition comprises from about 0.001% to about 10.0% pilocarpine (in whatever chosen form), preferably from about 0.005% to about 2.0%, and more preferably from about 0.01% to about 1.0%. In some embodiments, from about 0.07% to about 0.2% pilocarpine is used. One skilled in the art understands that the foregoing percentages will vary depending upon the particular source of pilocarpine utilized, the amount of pilocarpine desired in the final formulation, as well as on the particular release rate of pilocarpine desired. The buffer system of the inventive pilocarpine chewing gum composition provides for a final salivary pH in excess of at least about 7.5, preferably at least about 8.0, and more preferably from about 8.0 to about 10.0.

The inventive pilocarpine chewing gum compositions were made according to the following procedure. Silicon dioxide NF was passed through a #20 mesh screen, and then loaded into a blender containing mannitol granular USP and Pharmagum M. The material was blended for 10 minutes. Pilocarpine HCl USP was ground with the silicon dioxide using a mortar and pestle. The remaining silicon dioxide, along with magnesium stearate, was added into the mortar while continuing to grind. The ground materials were transferred into a plastic bag, and the mortar was rinsed using silicone dioxide and transferred into the bag. The contents of the bag were then blended for five minutes.

The blended mannitol gum base mixture was then added to the blended bag contents by continuous mixing until all the pilocarpine and gum base mixture had been blended together. Sodium carbonate, sodium bicarbonate, gum acacia, xanthan gum, and aspartame were then loaded into the blender along with natural and artificial flavors and blended for ten minutes with silicon dioxide. The flavors used were as follows: natural and artificial grape flavor, natural and artificial cherry flavor, natural and artificial fruit punch flavor, natural cherry flavor DURAROME®, and natural passion fruit flavor DURAROME®.

The blend was passed through a #12 mesh screen and then blended for an additional 15 minutes. Magnesium stearate was passed through a #20 mesh screen and added to the blend and blended for five minutes. The blend was collected and placed in plastic bags. Two silica gel desiccant bags were placed around the plastic bags to absorb ambient moisture. The blend was then compressed into tablets. By using the above-described procedure, the average particle size of the drug (i.e., pilocarpine) in the chewing gum is about 20 microns, as compared to a typical average drug particle size of from about 75 to about 100 microns. In addition, the average particle size of the drug in the chewing gum is less than or equal to the average particle size of the carrier ingredients (e.g., gum base, binders, etc.).

The inventive pilocarpine chewing gum compositions were made according to the formulations shown in Table 6. The unit weight for each chewing gum was 2550 mg. The chewing gum remained stable following buccal administration and produced a large sized cud upon mastication.

TABLE 6
Inventive Pilocarpine Chewing Gum Formulations.

| | 2 mg | 3.5 mg | 5 mg |
|---|---|---|---|
| | Amount | Amount | Amount |
| Ingredient | (mg) | (mg) | (mg) |
| Pilocarpine HCl USP | 2.0 | 3.5 | 5 |
| Mannitol USP | 165 | 163.5 | 162 |
| Silicon Dioxide NF | 33 | 33 | 33 |
| Sodium Carbonate USP | 22 | 22 | 22 |
| Sodium Bicarbonate | 114 | 114 | 114 |
| Gum Acacia NF | 86 | 86 | 86 |
| Xanthan Gum NF | 2.5 | 2.5 | 2.5 |
| Aspartame USP | 14.3 | 14.3 | 14.3 |
| Natural & Artificial Grape Flavor | 43 | 43 | 43 |
| Natural & Artificial Cherry Flavor | 21.5 | 21.5 | 21.5 |
| Natural and Artificial Fruit Punch Flavor | 36 | 36 | 36 |
| Natural Cherry Flavor Durarome | 43 | 43 | 43 |
| Natural Passion Fruit Flavor Durarome | 7 | 7 | 7 |
| Magnesium Stearate NF | 45.5 | 45.5 | 45.5 |
| Gum Base (Pharmagum ™ M)¹ | 1915.2 | 1915.2 | 1915.2 |
| Gum Base GRAS² | 26-30% | 26-30% | 26-30% |
| Isomalt EP³ | 30-33% | 30-33% | 30-33% |
| Mannitol USP | 20-38% | 20-38% | 20-38% |
| Sorbitol USP | 0-20% | 0-20% | 0-20% |
| Silicon Dioxide NF | 0-1% | 0-1% | 0-1% |
| Colloidal Silicon Dioxide NF | 0-1% | 0-1% | 0-1% |
| Total | 2550 | 2550 | 2550 |

¹The sum of the individual Gum Base components will equal 100% based upon the above-listed components.
²GRAS: Generally Recognized as Safe, 21 Code of Federal Regulations (CFR) 172.615.
³EP: European Pharmacopia.

The pilocarpine chewing gum compositions of the present invention can be used, e.g., for treatment of dry mouth caused by medication such as decongestants, diuretics, antidepressants, antihypertensives, and antihistamines; medical conditions such as autoimmune diseases (e.g., Sjögren's syndrome, rheumatoid arthritis), xerostomia, mucositis, or stomatotitis; or medical therapy such as radiotherapy for head and neck cancers. In certain instances, after the introduction of a serving size piece of the chewing gum composition into the mouth, the subject chews the chewing gum as is normally done with any non-medicated type of chewing gum for about 5 to about 20 minutes, at approximately an average rate of about 10 to about 45 chews per minute. The gum is then discarded.

A typical dosage form of the pilocarpine chewing gum of the present invention is designed to produce an average plasma concentration of at least from about 10 to about 100 nanograms of pilocarpine per milliliter of plasma. For example, a 5 mg pilocarpine chewing gum can be designed to produce a mean peak plasma concentration within the range of from about 10 to about 100 nanograms of pilocarpine per milliliter of plasma within about 5 minutes to about 2 hours.

The pilocarpine chewing gum compositions of the present invention provide a stable, convenient, reliable, practical, and painless system for delivering pilocarpine across the buccal mucosa. Notably, the chewing gum compositions are capable of rapidly delivering pilocarpine so that a therapeutically effective amount of pilocarpine enters the bloodstream within about 30 minutes, 20 minutes, 15 minutes, 10 minutes, 5 minutes, or even within about 1-2 minutes after pilocarpine is released from the gum base carrier.

### Example 3

### Pilocarpine Chewing Gum Saliva Output Studies

This example illustrates a comparison of the saliva output between an inventive pilocarpine chewing gum composition and a dose equivalent commercial oral tablet.

The salivary output was measured in a single dose two-way crossover study in 5 healthy normal subjects. The subjects were randomized to receive a single dose of either the inventive pilocarpine chewing gum or a dose equivalent commercial oral tablet (Salagen®) during each treatment period depending on their randomization sequence. The pilocarpine chewing gum was chewed for 30 minutes and the Salagen® tablet was swallowed with 240 ml water. Each treatment was separated by a washout period of 7 days. The subject had fasted overnight before reporting for the study and abstained from food or water during the study. Sialometric measurements were performed at 0 (pre-dose), 5, 15, 30, 60, and 120 minutes by collecting saliva from the left parotid duct using a pre-weighed patch held in place for 5 minutes.

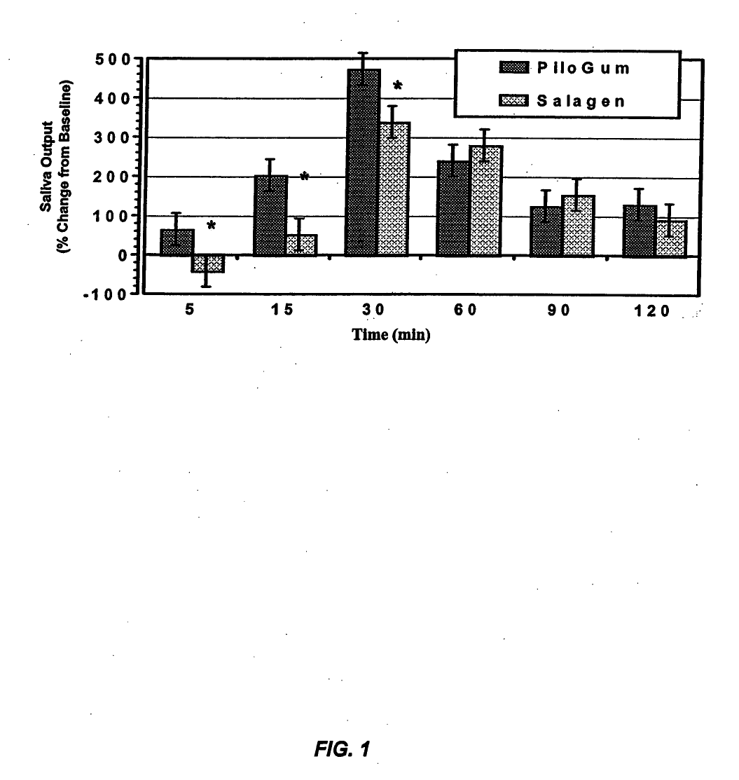

FIG. 1 shows the mean saliva output over time for a 5 mg pilocarpine chewing gum composition of the present invention as compared to a dose equivalent commercial oral tablet (Salagen®). The 5 mg pilocarpine chewing gum increased saliva production by about 50 to about 100 percent within about 5 minutes following administration, while the commercial oral tablet did not increase saliva production. Similarly, the 5 mg pilocarpine chewing gum increased saliva production by about 200 percent within about 15 minutes following administration, while the commercial oral tablet only increased saliva production by about 50%. Likewise, the 5 mg pilocarpine chewing gum increased saliva production by about 400 to about 500 percent within about 30 minutes following administration, while the commercial oral tablet only increased saliva production by about 300%. As such, this study illustrates that the inventive pilocarpine chewing gum compositions substantially increase the bioavailability of pilocarpine and reduce the time to onset of therapeutic activity as compared to a traditional dosage form for oral administration.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A solid dosage form composition for delivery of pilocarpine across the oral mucosa, said composition comprising:
(a) pilocarpine or a pharmaceutically acceptable salt thereof;
(b) a carrier; and
(c) a binary buffer system comprising a strong base and a weak base, wherein the amount of said strong base is less than the amount of said weak base,
wherein said binary buffer system raises the pH of saliva to a pH greater than about 7.5, irrespective of the starting pH of saliva.

2. A composition of claim 1, wherein the amount of said strong base is sufficiently less than the amount of said weak base to retain the solid dosage form for at least 3 months at room temperature.

3. A composition of claim 1, wherein said binary buffer system raises the pH of saliva to a pH of from about 8.0 to about 10.0, irrespective of the starting pH of saliva.

4. A composition of claim 1, wherein said pharmaceutically acceptable salt of pilocarpine is selected from the group consisting of pilocarpine hydrochloride, pilocarpine nitrate, pilocarpine sulfate, pilocarpine acetate, pilocarpine citrate, pilocarpine tartrate, pilocarpine zinc chloride monohydrate, pilocarpine salicylate, a concentrated extract of Pilocarpus leaves, and combinations thereof.

5. A composition of claim 1, wherein said strong base is a carbonate salt.

6. A composition of claim 5, wherein said carbonate salt is selected from the group consisting of sodium carbonate and potassium carbonate.

7. A composition of claim 1, wherein said weak base is a bicarbonate salt.

8. A composition of claim 7, wherein said bicarbonate salt is selected from the group consisting of sodium bicarbonate and potassium bicarbonate.

9. A composition of claim 1, wherein said strong base is sodium carbonate and said weak base is sodium bicarbonate.

10. A composition of claim 9, wherein the ratio of sodium carbonate to sodium bicarbonate is at least about 1:3 by weight.

11. A composition of claim 10, wherein the ratio of sodium carbonate to sodium bicarbonate is from about 1:4 to about 1:6 by weight.

12. A composition of claim 1, wherein said carrier is selected from the group consisting of a gum base, a binder, and combinations thereof.

13. A composition of claim 12, wherein said gum base comprises at least one hydrophobic polymer and at least one hydrophilic polymer.

14. A composition of claim 13, wherein said hydrophobic polymer is selected from the group consisting of a natural polymer, a synthetic polymer, and combinations thereof.

15. A composition of claim 14, wherein said synthetic polymer is selected from the group consisting of a butadiene-styrene copolymer, polyethylene, polyvinylester, butyl rubber, polyisobutylene, and combinations thereof.

16. A composition of claim 13, wherein said hydrophilic polymer is polyvinylacetate.

17. A composition of claim 12, wherein said gum base is selected from the group consisting of Pharmagum™ M, Pharmagum™ C, Pharmagum™ S, and combinations thereof.

18. A composition of claim 12, wherein said gum base is from about 40 to about 90 weight percent of the composition.

19. A composition of claim 18, wherein said gum base is from about 70 to about 80 weight percent of the composition.

20. A composition of claim 12, wherein said binder is selected from the group consisting of a sugar, a sugar alcohol, a natural gum, and combinations thereof.

21. A composition of claim 20, wherein said sugar alcohol is selected from the group consisting of mannitol, sorbitol, xylitol, and combinations thereof.

22. A composition of claim 20, wherein said natural gum is selected from the group consisting of acacia gum, xanthan gum, and combinations thereof.

23. A composition of claim 1, wherein said composition further comprises a sweetening agent, a flavoring agent, a protecting agent, a plasticizer, a wax, an elastomeric solvent, a filler material, a preservative, a lubricating agent, a wetting agent, an emulsifying agent, a solubilizing agent, a suspending agent, a coloring agent, a disintegrating agent, or combinations thereof.

24. A composition of claim 1, wherein said composition is a dosage form selected from the group consisting of a chewing gum, a lozenge, a chewable tablet, and a dissolving tablet.

25. A composition of claim 1, wherein said oral mucosa is selected from the group consisting of the buccal mucosa, the sublingual mucosa, and a combination thereof.

26. A composition of claim 1, wherein the average particle size of pilocarpine or a pharmaceutically acceptable salt thereof is less than or equal to the average particle size of said carrier.

27. A composition of claim 1, wherein said pharmaceutically acceptable salt of pilocarpine is pilocarpine hydrochloride and said binary buffer system comprises sodium carbonate and sodium bicarbonate.

28. A composition of claim 27, wherein said composition comprises from about 0.01 to about 1.0 weight percent pilocarpine hydrochloride; from about 0.1 to about 3.0 weight percent sodium carbonate; and from about 3.0 to about 6.0 weight percent sodium bicarbonate.

29. A composition of claim 28, wherein said composition comprises from about 0.07 to about 0.2 weight percent pilocarpine hydrochloride; about 0.85 weight percent sodium carbonate; and about 4.5 weight percent sodium bicarbonate.

30. A composition of claim 29, wherein said composition is administered buccally.

31. A composition of claim 29, wherein said composition is a chewing gum.

32. A composition of claim 31, wherein the weight of said chewing gum is from about 2000 to about 3000 mg.

33. A composition of claim 29, wherein said composition is stable upon storage for at least 3 months at 30° C.

34.-137. (canceled)
